SEQUENCE LISTING (46 sequences)

SEQ ID NO: 1 - protein, 112 aa, Artificial Sequence
  Peptide from human
DQQVKQNSPSLSVQEGRISILNCDYTNSMFDYFLWYKKYPAEGPTFLISISSIKDKNEDGRFTVFLNKSAKHLSLHIVPSQPGDSAVYFCAAPPPSGNQFYFGTGTSLTVIP

SEQ ID NO: 2 - protein, 116 aa, Artificial Sequence
  Peptide from human
DVKVTQSSRYLVKRTGEKVFLECVQDMDHENMFWYRQDPGLGLRLIYFSYDVKMKEKGDIPEGYSVSREKKERFSLILESASTNQTSMYLCASSTRLAGAQRDTQYFGPGTRLTVL

SEQ ID NO: 3 - DNA, 336 bp, Artificial Sequence
  DNA from human
gatcagcaggttaaacaaaatagcccgtcactttcagttcaagaaggacgcatctccatactcaactgcgattacacgaactctatgtttgattactttctttggtacaagaagtacccagcagagggtccgactttccttatctccataagcagtatcaaagataagaacgaagacggtcggttcacagtgttccttaacaaaagcgcaaagcacttgtctctgcacattgtgccgtcccaacccggcgactcagcagtgtacttctgcgccgcacccccaccctccggtaatcaattttacttcggtacggggacgtctctcactgttataccc

SEQ ID NO: 4 - DNA, 348 bp, Artificial Sequence
  DNA from human
gacgtgaaggtcactcagtcttcacggtatcttgttaagcgcactggcgaaaaagtttttctggagtgtgtccaggacatggatcacgaaaatatgttttggtatcgccaagaccctgggctgggtctcagactgatttactttagctacgacgttaaaatgaaggaaaagggtgatattcctgaagggtatagtgtttcacgcgagaagaaagagcggttctcactgatccttgagtcagcctccactaaccaaacatccatgtacctttgcgcatctagtacaaggctcgcgggcgctcaacgggatacccaatatttcggtccgggcacgcggttgaccgtcctg

SEQ ID NO: 5 - protein, 111 aa, Artificial Sequence
  Peptide from human
EDVEQSLFLSVREGDSSVINCTYTDSSSTYLYWYKQEPGAGLQLLTYIFSNMDMKQDQRLTVLLNKKDKHLSLRIADTQTGDSAIYFCAESIPSGYNKLIFGAGTRLAVHP

SEQ ID NO: 6 - protein, 113 aa, Artificial Sequence
  Peptide from human
GVAQSPRYKIIEKRQSVAFWCNPISGHATLYWYQQILGQGPKLLIQFQNNGVVDDSQLPKDRFSAERLKGVDSTLKIQPAKLEDSAVYLCASSLGLAGEDTQYFGPGTRLTVL

SEQ ID NO: 7 - DNA, 333 bp, Artificial Sequence
  DNA from human
gaggacgtagaacaatctctttttctctctgtccgcgagggagattcctctgtaatcaattgtacttacaccgatagcagctcaacgtacctctactggtataagcaggagcccggcgcaggcttgcaacttctgacttatatatttagcaatatggatatgaaacaagaccaacgattgacagtgcttctgaacaagaaagacaagcacctttctcttcgcattgcggacacccagacaggggactcagcaatttatttttgcgctgaaagcattccctctggatacaataagttgatatttggagcggggaccaggctggcggtccatcca

SEQ ID NO: 8 - DNA, 339 bp, Artificial Sequence
  DNA from human
ggtgtggcacaatcccccaggtacaagatcatcgagaaaaggcagtccgtcgcgttttggtgtaatccaatatctggacatgccacactttactggtatcaacagatactgggtcaggggccgaagctgcttatacagtttcaaaacaatggcgtggtcgatgacagtcagcttcccaaagacagattttctgcagagagactgaagggggtggatagtaccttgaaaatacaaccggcgaaactcgaagacagtgctgtctacctttgcgcttcctcactcggattggctggagaggacacacaatactttggtccaggaaccagactcacggttctg

SEQ ID NO: 9 - protein, 119 aa, Artificial Sequence
  Peptide from human
QQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAAKLPQAAGNKLTFGGGTRVLVKP

SEQ ID NO: 10 - protein, 115 aa, Artificial Sequence
  Peptide from human
MVIQNPRYQVTQFGKPVTLSCSQTLNHNVMYWYQQKSSQAPKLLFHYYDKDFNNEADTPDNFQSRRPNTSFCFLDIRSPGLGDAAMYLCATSSSIAGDTRNNEQFFGPGTRLTVL

SEQ ID NO: 11 - DNA, 357 bp, Artificial Sequence
  DNA from human
caacagaagaatgacgatcaacaagtaaagcaaaacagcccctctctttcagtacaagaaggacgcatctctatacttaactgcgactatacaaatagcatgtttgactacttcctgtggtacaaaaaatatccagcagagggtcccaccttccttatcagcataagtagcattaaagacaaaaatgaagacggtaggttcacagtcttcctcaataaaagcgctaaacatctctcactccatattgtaccgtcccaacctggtgacagtgcggtatatttttgcgcagccaaacttcctcaggcggctgggaataaactgacgtttggcggcgggacgagggtcctggtaaaaccc

SEQ ID NO: 12 - DNA, 345 bp, Artificial Sequence
  DNA from human
atggttatccaaaacccaagataccaggttacgcagttcggcaagccagtaacactttcatgttctcaaaccttgaatcacaatgtcatgtactggtatcaacaaaagtcaagccaggccccaaaacttctctttcactattacgataaagacttcaacaatgaggccgatacccccgacaactttcagagtaggaggcccaacacaagtttttgtttcttggacatccgctcacccggacttggcgatgcggcgatgtatctctgtgcgacatcaagtagtatagcgggggatacgcggaacaacgaacaattttttggtcctggaaccaggcttacggtattg

SEQ ID NO: 13 - protein, 137 aa, Homo sapiens
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 14 - protein, 26 aa, Artificial Sequence
  Signal peptide
MATGSRTSLLLAFGLLCLPWLQEASA

SEQ ID NO: 15 - protein, 26 aa, Artificial Sequence
  Signal peptide
MATGSRTSLLLAFGLLCLPCLQEGSA

SEQ ID NO: 16 - protein, 9 aa, Artificial Sequence
  Peptide epitope
LLWTLVVLL

SEQ ID NO: 17 - protein, 6 aa, Artificial Sequence
  Peptide from human
NSMFDY

SEQ ID NO: 18 - protein, 7 aa, Artificial Sequence
  Peptide from human
ISSIKDK

SEQ ID NO: 19 - protein, 12 aa, Artificial Sequence
  Peptide from human
CAAPPPSGNQFY

SEQ ID NO: 20 - protein, 5 aa, Artificial Sequence
  Peptide from human
MDHEN

SEQ ID NO: 21 - protein, 6 aa, Artificial Sequence
  Peptide from human
SYDVKM

SEQ ID NO: 22 - protein, 15 aa, Artificial Sequence
  Peptide from human
ASSTRLAGAQRDTQY

SEQ ID NO: 23 - protein, 6 aa, Artificial Sequence
  Peptide from human
DSSSTY

SEQ ID NO: 24 - protein, 7 aa, Artificial Sequence
  Peptide from human
IFSNMDM

SEQ ID NO: 25 - protein, 12 aa, Artificial Sequence
  Peptide from human
AESIPSGYNKLI

SEQ ID NO: 26 - protein, 5 aa, Artificial Sequence
  Peptide from human
SGHAT

SEQ ID NO: 27 - protein, 6 aa, Artificial Sequence
  Peptide from human
FQNNGV

SEQ ID NO: 28 - protein, 13 aa, Artificial Sequence
  Peptide from human
ASSLGLAGEDTQY

SEQ ID NO: 29 - protein, 6 aa, Artificial Sequence
  Peptide from human
NSMFDY

SEQ ID NO: 30 - protein, 7 aa, Artificial Sequence
  Peptide from human
ISSIKDK

SEQ ID NO: 31 - protein, 13 aa, Artificial Sequence
  Peptide from human
AAKLPQAAGNKLT

SEQ ID NO: 32 - protein, 5 aa, Artificial Sequence
  Peptide from human
LNHNV

SEQ ID NO: 33 - protein, 6 aa, Artificial Sequence
  Peptide from human
YYDKDF

SEQ ID NO: 34 - protein, 16 aa, Artificial Sequence
  Peptide from human
ATSSSIAGDTRNNEQF

SEQ ID NO: 35 - protein, 249 aa, Artificial Sequence
  Peptide from human/mouse
DQQVKQNSPSLSVQEGRISILNCDYTNSMFDYFLWYKKYPAEGPTFLISISSIKDKNEDGRFTVFLNKSAKHLSLHIVPSQPGDSAVYFCAAPPPSGNQFYFGTGTSLTVIPDIQNPEPAVYQLKDPRSQDSTLCLFTDFDSQINVPKTMESGTFITDKTVLDMKAMDSKSNGAIAWSNQTSFTCQDIFKETNACYPSSDVPCDATLTEKSFETDMNLNFQNLSVMGLRILLLKVAGFNLLMTLRLWSS

SEQ ID NO: 36 - protein, 289 aa, Artificial Sequence
  Peptide from human/mouse
DVKVTQSSRYLVKRTGEKVFLECVQDMDHENMFWYRQDPGLGLRLIYFSYDVKMKEKGDIPEGYSVSREKKERFSLILESASTNQTSMYLCASSTRLAGAQRDTQYFGPGTRLTVLEDLRNVTPPKVSLFEPSKAEIANKQKATLVCLARGFFPDHVELSWWVNGKEVHSGVSTDPQAYKESNYSYCLSSRLRVCATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 37 - protein, 248 aa, Artificial Sequence
  Peptide from human/mouse
EDVEQSLFLSVREGDSSVINCTYTDSSSTYLYWYKQEPGAGLQLLTYIFSNMDMKQDQRLTVLLNKKDKHLSLRIADTQTGDSAIYFCAESIPSGYNKLIFGAGTRLAVHPDIQNPEPAVYQLKDPRSQDSTLCLFTDFDSQINVPKTMESGTFITDKTVLDMKAMDSKSNGAIAWSNQTSFTCQDIFKETNACYPSSDVPCDATLTEKSFETDMNLNFQNLSVMGLRILLLKVAGFNLLMTLRLWSS

SEQ ID NO: 38 - protein, 286 aa, Artificial Sequence
  Peptide from human/mouse
GVAQSPRYKIIEKRQSVAFWCNPISGHATLYWYQQILGQGPKLLIQFQNNGVVDDSQLPKDRFSAERLKGVDSTLKIQPAKLEDSAVYLCASSLGLAGEDTQYFGPGTRLTVLEDLRNVTPPKVSLFEPSKAEIANKQKATLVCLARGFFPDHVELSWWVNGKEVHSGVSTDPQAYKESNYSYCLSSRLRVCATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 39 - protein, 256 aa, Artificial Sequence
  Peptide from human/mouse
QQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAAKLPQAAGNKLTFGGGTRVLVKPDIQNPEPAVYQLKDPRSQDSTLCLFTDFDSQINVPKTMESGTFITDKTVLDMKAMDSKSNGAIAWSNQTSFTCQDIFKETNACYPSSDVPCDATLTEKSFETDMNLNFQNLSVMGLRILLLKVAGFNLLMTLRLWSS

SEQ ID NO: 40 - protein, 288 aa, Artificial Sequence
  Peptide from human/mouse
MVIQNPRYQVTQFGKPVTLSCSQTLNHNVMYWYQQKSSQAPKLLFHYYDKDFNNEADTPDNFQSRRPNTSFCFLDIRSPGLGDAAMYLCATSSSIAGDTRNNEQFFGPGTRLTVLEDLRNVTPPKVSLFEPSKAEIANKQKATLVCLARGFFPDHVELSWWVNGKEVHSGVSTDPQAYKESNYSYCLSSRLRVCATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 41 - DNA, 747 bp, Artificial Sequence
  DNA from human/mouse
gatcagcaggttaaacaaaatagcccgtcactttcagttcaagaaggacgcatctccatactcaactgcgattacacgaactctatgtttgattactttctttggtacaagaagtacccagcagagggtccgactttccttatctccataagcagtatcaaagataagaacgaagacggtcggttcacagtgttccttaacaaaagcgcaaagcacttgtctctgcacattgtgccgtcccaacccggcgactcagcagtgtacttctgcgccgcacccccaccctccggtaatcaattttacttcggtacggggacgtctctcactgttatacccgacatccagaatccagagccagccgtgtaccagctgaaggaccctaggagccaggactctaccctctgcctgttcaccgacttcgacagccagatcaacgtgcccaagacaatggagagcggcaccttcatcaccgacaagaccgtgctggacatgaaggctatggacagcaagagcaacggagccatcgcttggagcaaccagaccagcttcacttgccaggacatcttcaaggagaccaacgcttgctacccctctagcgacgtgccttgcgacgccacactgacagagaagagcttcgagaccgacatgaacctgaacttccagaacctgagcgtgatgggcctgagaatcctgctgctgaaggtggccggattcaacctgctgatgaccctgaggctgtggtcttcc

SEQ ID NO: 42 - DNA, 867 bp, Artificial Sequence
  DNA from human/mouse
gacgtgaaggtcactcagtcttcacggtatcttgttaagcgcactggcgaaaaagtttttctggagtgtgtccaggacatggatcacgaaaatatgttttggtatcgccaagaccctgggctgggtctcagactgatttactttagctacgacgttaaaatgaaggaaaagggtgatattcctgaagggtatagtgtttcacgcgagaagaaagagcggttctcactgatccttgagtcagcctccactaaccaaacatccatgtacctttgcgcatctagtacaaggctcgcgggcgctcaacgggatacccaatatttcggtccgggcacgcggttgaccgtcctggaggacctacgtaacgtgacccctcccaaggtgtccctgttcgagcctagcaaggccgagatcgccaacaagcagaaggccacactcgtctgcctggctagaggcttcttcccagaccacgtggagctgtcttggtgggtgaacggcaaggaggtgcactcaggagtgtctaccgaccctcaggcctacaaggagagcaactacagctactgcctgtcctccagactcagggtctgcgccaccttttggcacaaccctcggaaccacttccgctgtcaggtccagttccacggcctgtccgaggaagacaagtggccagagggctctcctaagccagtgacacagaacatcagcgccgaggcttggggaagagccgattgcggaatcaccagcgcctcttaccagcagggagtgctgtcagctaccatcctgtacgagatcctgctgggcaaggccacactgtacgcagtgctggtgtccactctggtcgtgatggctatggtgaagcggaagaacagc

SEQ ID NO: 43 - DNA, 744 bp, Artificial Sequence
  DNA from human/mouse
gaggacgtagaacaatctctttttctctctgtccgcgagggagattcctctgtaatcaattgtacttacaccgatagcagctcaacgtacctctactggtataagcaggagcccggcgcaggcttgcaacttctgacttatatatttagcaatatggatatgaaacaagaccaacgattgacagtgcttctgaacaagaaagacaagcacctttctcttcgcattgcggacacccagacaggggactcagcaatttatttttgcgctgaaagcattccctctggatacaataagttgatatttggagcggggaccaggctggcggtccatccagacatccagaatccagagccagccgtgtaccagctgaaggaccctaggagccaggactctaccctctgcctgttcaccgacttcgacagccagatcaacgtgcccaagacaatggagagcggcaccttcatcaccgacaagaccgtgctggacatgaaggctatggacagcaagagcaacggagccatcgcttggagcaaccagaccagcttcacttgccaggacatcttcaaggagaccaacgcttgctacccctctagcgacgtgccttgcgacgccacactgacagagaagagcttcgagaccgacatgaacctgaacttccagaacctgagcgtgatgggcctgagaatcctgctgctgaaggtggccggattcaacctgctgatgaccctgaggctgtggtcttcc

SEQ ID NO: 44 - DNA, 858 bp, Artificial Sequence
  DNA from human/mouse
ggtgtggcacaatcccccaggtacaagatcatcgagaaaaggcagtccgtcgcgttttggtgtaatccaatatctggacatgccacactttactggtatcaacagatactgggtcaggggccgaagctgcttatacagtttcaaaacaatggcgtggtcgatgacagtcagcttcccaaagacagattttctgcagagagactgaagggggtggatagtaccttgaaaatacaaccggcgaaactcgaagacagtgctgtctacctttgcgcttcctcactcggattggctggagaggacacacaatactttggtccaggaaccagactcacggttctggaggacctacgtaacgtgacccctcccaaggtgtccctgttcgagcctagcaaggccgagatcgccaacaagcagaaggccacactcgtctgcctggctagaggcttcttcccagaccacgtggagctgtcttggtgggtgaacggcaaggaggtgcactcaggagtgtctaccgaccctcaggcctacaaggagagcaactacagctactgcctgtcctccagactcagggtctgcgccaccttttggcacaaccctcggaaccacttccgctgtcaggtccagttccacggcctgtccgaggaagacaagtggccagagggctctcctaagccagtgacacagaacatcagcgccgaggcttggggaagagccgattgcggaatcaccagcgcctcttaccagcagggagtgctgtcagctaccatcctgtacgagatcctgctgggcaaggccacactgtacgcagtgctggtgtccactctggtcgtgatggctatggtgaagcggaagaacagc

SEQ ID NO: 45 - DNA, 768 bp, Artificial Sequence
  DNA from human/mouse
caacagaagaatgacgatcaacaagtaaagcaaaacagcccctctctttcagtacaagaaggacgcatctctatacttaactgcgactatacaaatagcatgtttgactacttcctgtggtacaaaaaatatccagcagagggtcccaccttccttatcagcataagtagcattaaagacaaaaatgaagacggtaggttcacagtcttcctcaataaaagcgctaaacatctctcactccatattgtaccgtcccaacctggtgacagtgcggtatatttttgcgcagccaaacttcctcaggcggctgggaataaactgacgtttggcggcgggacgagggtcctggtaaaacccgacatccagaatccagagccagccgtgtaccagctgaaggaccctaggagccaggactctaccctctgcctgttcaccgacttcgacagccagatcaacgtgcccaagacaatggagagcggcaccttcatcaccgacaagaccgtgctggacatgaaggctatggacagcaagagcaacggagccatcgcttggagcaaccagaccagcttcacttgccaggacatcttcaaggagaccaacgcttgctacccctctagcgacgtgccttgcgacgccacactgacagagaagagcttcgagaccgacatgaacctgaacttccagaacctgagcgtgatgggcctgagaatcctgctgctgaaggtggccggattcaacctgctgatgaccctgaggctgtggtcttcc

SEQ ID NO: 46 - DNA, 864 bp, Artificial Sequence
  DNA from human/mouse
atggttatccaaaacccaagataccaggttacgcagttcggcaagccagtaacactttcatgttctcaaaccttgaatcacaatgtcatgtactggtatcaacaaaagtcaagccaggccccaaaacttctctttcactattacgataaagacttcaacaatgaggccgatacccccgacaactttcagagtaggaggcccaacacaagtttttgtttcttggacatccgctcacccggacttggcgatgcggcgatgtatctctgtgcgacatcaagtagtatagcgggggatacgcggaacaacgaacaattttttggtcctggaaccaggcttacggtattggaggacctacgtaacgtgacccctcccaaggtgtccctgttcgagcctagcaaggccgagatcgccaacaagcagaaggccacactcgtctgcctggctagaggcttcttcccagaccacgtggagctgtcttggtgggtgaacggcaaggaggtgcactcaggagtgtctaccgaccctcaggcctacaaggagagcaactacagctactgcctgtcctccagactcagggtctgcgccaccttttggcacaaccctcggaaccacttccgctgtcaggtccagttccacggcctgtccgaggaagacaagtggccagagggctctcctaagccagtgacacagaacatcagcgccgaggcttggggaagagccgattgcggaatcaccagcgcctcttaccagcagggagtgctgtcagctaccatcctgtacgagatcctgctgggcaaggccacactgtacgcagtgctggtgtccactctggtcgtgatggctatggtgaagcggaagaacagc

(19) United States
(12) Patent Application Publication
(10) Pub. No.: US 2021/0369776 A1
(43) Pub. Date: Dec. 2, 2021

(54) COMBINATIONAL TCR-T CELL THERAPY TARGETING TUMOR ANTIGENS, TGF-BETA, AND IMMUNE CHECKPOINTS

(71) Applicants: GUANGDONG TCRCURE BIOPHARMA TECHNOLOGY CO., LTD., Guangzhou, Guangdong (CN); TCRCURE BIOPHARMA CORP., Chapel Hill, NC (US)

(72) Inventors: Si Li, Alhambra, CA (US); Pin Wang, Los Angeles, CA (US); Paul Bryson, Culver City, CA (US); Peter Alexander, Durham, NC (US); Rui Chen, Durham, NC (US)

(21) Appl. No.: 17/311,114

(22) PCT Filed: Dec. 5, 2019
(86) PCT No.: PCT/US2019/064757

§ 371 Date: Jun. 4, 2021

Related U.S. Application Data
(60) Provisional application No. 62/776,012, filed on Dec. 6, 2018.

(51) Int. Cl. A61K 35/17; C07K 14/725; C07K 16/30; A61K 45/06

(52) U.S. Cl. CPC A61K 35/17; C07K 14/7051; C07K 16/30; C07K 2317/34; C07K 2319/30; C07K 2317/565; A61K 45/06

(57) ABSTRACT

The present disclosure is directed towards genetically engineered TCR-T cells to recognize tumor antigens and simultaneously secrete a binding protein that blocks an immune checkpoint molecule and TGF-beta. These engineered T cells demonstrate stronger antitumor response and reduced T cell exhaustion. The present disclosure provides immunotherapy against HPV- or EBV-positive cancers, among others.

### CLAIM OF PRIORITY

This application claims the benefit of U.S. Provisional Application No. 62/776,012, filed on Dec. 6, 2018. The entire contents of the foregoing are incorporated herein by reference.

### TECHNICAL FIELD

The present disclosure generally relates to engineered cells and compositions thereof, particularly, T cells comprising genetically engineered T Cell receptors (TCRs), TGF-β receptors (e.g., TGF-β trap) and checkpoint inhibitors (CPIs). Methods for using the compositions to treat cancer are also disclosed herein.

### BACKGROUND OF THE INVENTION

In proliferating infected B cells, Epstein-Barr virus (EBV) installs a program of gene expression, the “growth” or “latency III” program. This type of latency is found in in vitro EBV-induced lymphoblastoid cell lines (LCLs), in post-transplant lymphoproliferative diseases (Brink A A, 1997, J Clin Pathol 50: 911-918), as well as in EBV-infected B cells in lymphoid organs during primary and persistent EBV infection, where this program is thought to lead to amplification of EBV load through proliferation of infected cells (Young L S, 2004, Nat Rev Cancer 4: 757-768; Hochberg D, 2004, Proc Natl Acad Sci USA 101: 239-244). Several immunogenic EBV antigens, the latent membrane proteins (LMP1, LMP2A, LMP2B) and the Epstein-Barr nuclear antigens (EBNA1, -2, -3A, -3B, -3C, -LP), are expressed in latency III EBV-infected B cells. Epstein-Barr virus (EBV) DNA is found in patients with nasopharyngeal cancer (Mutirangura et al., Clin Cancer Res. 4: 665-9 (1998); Lo et al., Cancer Res. 59: 1188-91 (1999)), certain lymphomas (Lei et al., Br J Haematol. 111:239-46 (2000); Gallagher et al., Int J Cancer. 84: 442-8 (1999); Dronet et al., J Med Viral. 57: 383-9 (1999)), breast cancer (Bonnet, M. et al., J. Natl. Cancer Inst., 91: 1376-1381 (1999)) and hepatocellular carcinoma (Sugawara, Y. et al., Virology, 256: 196-202 (1999)).

Adoptive cell transfer (ACT), as a modality of immunotherapy for cancer, has demonstrated remarkable success in treating hematologic malignancies and malignant melanoma. One form of ACT, which uses genetically modified T cells expressing a chimeric antigen receptor (CAR) to specifically target a tumor-associated-antigen (TAA), such as CD19 or GD2, has displayed encouraging results in clinical trials for treating such diseases as B cell malignancies.

Despite the documented success of CAR-T cell therapy in patients with hematologic malignancies, only modest responses have been observed in solid tumors. This can be attributed, in part, to the establishment of an immunosuppressive tumor microenvironment. Such milieu involves the upregulation of several intrinsic inhibitory pathways mediated by increased expression of inhibitory receptors (IRs) in T cells reacting with their cognate ligands within the tumor (Ping Y, et al, Protein Cell 2018, 9(3):254-266). In addition, unlike naturally occurring T cell receptors (TCRs), CARs can directly and selectively recognize cell surface TAAs in a major histocompatibility class (MHC)-independent manner. The high density of TAAs could affect the solid tumor penetration by CAR-T cells. However, due to the scarcity of targeted MHC-dependent antigens on the cancer cell surface, T cells with genetically engineered TCRs mimicking natural TCRs can penetrate much deeper than CAR-T cells. A TCR may recognize either intracellular or extracellular antigen in the context of MHC. When designing a TCR to target tumor, having the option to target intracellular tumor antigen may be advantageous. (Fesnak A D, et al. Nat Rev Cancer. 2016 Aug. 23; 16(9): 566-581.)

So far, several IRs have been characterized in T cells, such as CTLA-4, T cell Ig mucin-3 (TIM-3), lymphocyte-activation gene 3 (LAG-3), and programmed death-1 (PD-1). These molecules are upregulated following sustained activation of T cells in chronic diseases and cancer, and they promote T cell dysfunction and exhaustion, thus resulting in tumoral escape from immune surveillance. Unlike other IRs, PD-1 is upregulated shortly after T cell activation, which in turn inhibits T cell effector function via interacting with one of its two ligands: PD-L1 or PD-L2. PD-L1 is constitutively expressed on T cells, B cells, macrophages, and dendritic cells (DCs). It is also shown to be abundantly expressed in a wide variety of solid tumors. In contrast, the expression of PD-L1 in normal tissues is undetectable. As a consequence of its critical role in immunosuppression, PD-1 has been the focus of recent research, aiming to neutralize its negative effect on T cells and enhance antitumor responses. Clinical studies have demonstrated that PD-1 blockade significantly mediates tumor regression in colorectal, renal and lung cancers and melanoma. (Chae Y K, et al, J Immunother Cancer. 2018; 6: 39; Le D T, et al. N Engl J Med 2015; 372: 2509-20.)

Both the TGFβ ligand and its receptor have been studied intensively as therapeutic targets. There are three ligand isoforms, TGFβ1, 2 and 3, all of which exist as homodimers. There are also three TGFβ receptors (TGFβR), which are called TGFβR type I, II and III (López-Casillas et al., J. Cell Biol. 1994; 124:557-68). TGFβRI is the signaling chain and cannot bind ligand. TGFβRII binds the ligand TGFβ1 and 3, but not TGFβ2, with high affinity. The TGFβRII/TGFβ complex recruits TGFβRI to form the signaling complex (Won et al., Cancer Res. 1999: 59:1273-7). TGFβRIII is a positive regulator of TGFβ binding to its signaling receptors and binds all 3 TGFβ isoforms with high affinity. On the cell surface, the TGFβ/TGFβRIII complex binds TGFβRII and then recruits TGFβRI, which displaces TGFβRIII to form the signaling complex.

Although the three different TGFβ isoforms all signal through the same receptor, they are known to have differential expression patterns and non-overlapping functions in vivo. The three different TGFβ isoform knockout mice have distinct phenotypes, indicating numerous non-compensated functions (Bujak et al., Cardiovasc Res. 2007: 74: 184-95). Therefore, given the predominant roles of TGFβ1 and TGFβ2 in the tumor microenvironment and cardiac physiology, respectively, a therapeutic agent that neutralizes TGFβ1 but not TGFβ2 could provide an optimal therapeutic index by minimizing the cardiotoxicity without compromising the anti-tumor activity.

Despite the promising clinical activities of immune checkpoint inhibitors so far, increasing the therapeutic index, either by increasing therapeutic efficacy or decreasing toxicity, or both, remains a central goal in the development of anticancer immunotherapeutics.

### SUMMARY OF THE INVENTION

The present disclosure provides an engineered T cell, comprising: a nucleic acid encoding an anti-LMP2 TCR wherein the anti-LMP2 TCR is a genetically engineered T cell receptor (TCR) that specifically binds to LMP2 in a tumor.

In an aspect of the invention, the anti-LMP2 TCR comprises the following motif sequences: alpha chain CDR1 (position 27-32), CDR2 (position 50-56), CDR3 (position 90-101) of amino acid SEQ ID NO:1 and beta chain CDR1 (position 27-31), CDR2 (position 49-54), CDR3 (position 92-106) of amino acid SEQ ID NO:2 respectively. In another embodiment, the anti-LMP2 TCR comprises an alpha chain variable domain of SEQ ID NO:1 and a beta chain variable domain of SEQ ID NO:2. In more embodiments, the nucleic acid encoding the genetically engineered TCR comprises the sequences set forth in SEQ ID NO:3 and SEQ ID NO:4.

In another aspect of the invention, the anti-LMP2 TCR comprises alpha chain CDR1 (position 25-30), CDR2 (position 48-54), CDR3 (position 89-100) of amino acid SEQ ID NO:5 and beta chain CDR1 (position 25-29), CDR2 (position 47-52), CDR3 (position 91-103) of amino acid SEQ ID NO:6 respectively. In more embodiments, the anti-LMP2 TCR comprises an alpha chain variable domain of SEQ ID NO:5 and a beta chain variable domain of SEQ ID NO:6. In preferred embodiments, the nucleic acid encoding the genetically engineered TCR comprises the sequences set forth in the SEQ ID NO:7 and SEQ ID NO:8.

In another aspect of the invention, the anti-LMP2 TCR comprises an alpha chain CDR1 (position 32-37), CDR2 (position 55-61), CDR3 (position 96-108) of amino acid SEQ ID NO:9 and beta chain CDR1 (position 25-29), CDR2 (position 47-52), CDR3 (position 90-105) of amino acid SEQ ID NO:10 respectively. In more embodiments, the anti-LMP2 TCR comprises an alpha chain variable domain of SEQ ID NO:9 and a beta chain variable domain of SEQ ID NO:10. In preferred embodiments, the nucleic acid encoding the genetically engineered TCR comprises the sequences set forth in the SEQ ID NO:11 and SEQ ID NO:12.

In another aspect of the invention, the anti-LMP2 TCR is constitutively expressed.

In another aspect of the invention, the engineered T cell further comprises an inhibitory protein that reduces function or expression of inhibitory receptors in a tumor.

In some embodiments, the inhibitory protein is an immune checkpoint inhibitor.

In some embodiments, the inhibitory protein blocks Programmed Cell Death Protein 1 (PD-1), wherein the protein is a single chain antibody (scFv). In preferred embodiments, the inhibitory protein is constitutively expressed.

In an aspect of the invention, a pharmaceutical composition comprising the supra mentioned engineered T cells and a pharmaceutically acceptable carrier is provided. Also, a method for treating cancer comprising administering to a subject in need thereof, a therapeutically effective amount of the pharmaceutical composition is provided, wherein the cancer is a nasopharyngeal carcinoma, Hodgkin's lymphoma, Burkitt's lymphoma, or stomach cancer.

In some embodiments, the method further comprises administering to the subject a therapeutically effective amount of an existing therapy comprising chemotherapy or radiation. In some embodiments, the cell and the existing therapy are administered sequentially or simultaneously.

The present invention also provides an engineered T cell, comprising: a nucleic acid encoding (a) a genetically engineered T cell receptor that specifically binds to an antigen in a tumor; (b) an inhibitory protein that reduces function or expression of an immune checkpoint in a tumor; and (c) a protein that binds to a member of the transforming growth factor beta (TGF-β) family. These engineered T cells demonstrate reduced T cell exhaustion; they thus have the capacity to induce a stronger anti-tumor response. The targeted transforming growth factor beta (TGF-β) can be TGF-β1, 2 or 3.

In an aspect of the invention, the immune checkpoint comprises one or more of PD1, PD-L1 and CTLA-4. In some embodiments, the inhibitory protein blocks Programmed Cell Death Protein 1 (PD-1), wherein the protein is a single chain antibody (scFv).

In an aspect of the invention, the tumor antigen is a human papillomavirus (HPV) or Epstein-Barr virus (EBV) antigen. In some embodiments, the genetically engineered T cell receptor is an anti-LMP2 TCR. In some embodiments, the anti-LMP2 TCR comprises an alpha chain variable domain selected from the group consisting of SEQ ID NO:1, 5 or 9 and a beta chain variable domain selected from the group consisting of SEQ ID NO:2, 6 or 10. In some embodiments, the nucleic acid encoding the anti-LMP2 TCR comprises SEQ ID NO:3 and SEQ ID NO:4. In some embodiments, the nucleic acid encoding the anti-LMP2 TCR comprises SEQ ID NO:7 and SEQ ID NO:8. In some embodiments, the nucleic acid encoding the anti-LMP2 TCR comprises SEQ ID NO:11 and SEQ ID NO:12. In some embodiments, the genetically engineered T cell receptor is an anti-E6 or anti-E7 TCR.

In another aspect of the invention, the genetically engineered TCR is constitutively expressed.

In an aspect of the invention, the binding protein targeting a member of the transforming growth factor beta family comprises a fragment of human TGFβRII. In one embodiment, the binding protein comprises the extracellular domain (ECD) of TGFβRII (SEQ ID NO: 13).

In an aspect of the invention, the inhibitory protein and/or TGFβ binding protein is constitutively expressed.

The present invention further provides a vector comprising the supra mentioned nucleic acid comprising (a) a nucleic acid encoding a genetically engineered T cell receptor that specifically binds to an antigen in a tumor; (b) a nucleic acid encoding an inhibitory protein that reduces function or expression of an immune checkpoint in a tumor; and (c) a nucleic acid encoding a protein that binds to a member of the transforming growth factor beta (TGF-β) family, wherein the vector is preferably a retroviral vector.

In an aspect of the invention, a pharmaceutical composition comprising the supra mentioned engineered T cells and a pharmaceutically acceptable carrier is provided. Also, a method for treating cancer comprising administering to a subject in need thereof, a therapeutically effective amount of the pharmaceutical composition is provided, wherein the cancer is predominantly a virus-associated malignancy.

In some embodiments, the cancer is an HPV or EBV positive cancer. In some embodiments, an EBV associated cancer can be but no limited a nasopharyngeal carcinoma, Hodgkin's lymphoma, Burkitt's lymphoma, or stomach cancer. In some embodiments, an HPV associated cancer can be, but not limited to cervical, anal, oropharyngeal, or reproductive organ cancers.

In an aspect of the invention, the tumor is a virus-associated tumor or tumor associated with viral onco-genes.

In some embodiments, the method further comprises administering to the subject a therapeutically effective amount of an existing therapy comprising chemotherapy or radiation. In some embodiments, the cell and the existing therapy are administered sequentially or simultaneously.

In another aspect of the invention, a method of producing a genetically engineered T cell comprises introducing a vector containing three transgenes: (1) the alpha chain of a genetically engineered T cell receptor that specifically binds to an antigen in a tumor, (2) the beta chain of same TCR, and (3) the variable regions of the heavy and light chain of a novel immune checkpoint inhibitor (ICI) linked with a GS linker, fused to a ligand-binding sequence of the extracellular domain of TCRβRII via a flexible linker peptide at the C terminus of the variable heavy chain, wherein the vector includes, but not limited to a retroviral vector. In more embodiments, the three transgenes are linked by 2A sequences. In some embodiments, the genetically engineered TCR further comprises a signal peptide sequence.

In one aspect, the disclosure is related to a T cell receptor (TCR) or antigen-binding fragment thereof, comprising an alpha chain including a variable alpha (Va) region and a beta chain comprising a variable beta (Vb) region.

In some embodiments, provided herein is the TCR or antigen-binding fragment that:

(1) the Va region comprises a complementarity determining region 1 (CDR1), a complementarity determining region 2 (CDR2), and a complementarity determining region 3 (CDR3), comprising CDR1, CDR2, and CDR3 of SEQ ID NO:1, respectively, and the Vb region comprises a CDR1, a CDR2, and a CDR3, comprising CDR1, CDR2, and CDR3 of SEQ ID NO: 2, respectively;

(2) the Va region comprises a CDR1, a CDR2, and a CDR3, comprising CDR1, CDR2, CDR3 of SEQ ID NO: 5, respectively, and the Vb region comprises a CDR1, a CDR2, and a CDR3, comprising the amino acid sequences of CDR1, CDR2, and CDR3 of SEQ ID NO: 6, respectively; or

(3) the Va region comprises a CDR1, a CDR2, and a CDR3, comprising CDR1, CDR2, CDR3 of SEQ ID NO: 9, respectively, and the Vb region comprises a CDR1, a CDR2, and a CDR3, comprising the amino acid sequences of CDR1, CDR2, and CDR3 of SEQ ID NO: 10, respectively.

In some embodiments, provided herein are the TCR or antigen-binding fragment that:

(1) the Va region comprises a CDR1, a CDR2, and a CDR3, comprising amino acids of SEQ ID NOs: 17-19, respectively, and the Vb region comprises a CDR1, a CDR2, and a CDR3, comprising amino acids of SEQ ID NOs: 20-22, respectively;

(2) the Va region comprises a CDR1, a CDR2, and a CDR3, comprising amino acids of SEQ ID NOs: 23-25, respectively, and the Vb region comprises a CDR1, a CDR2, and a CDR3, comprising amino acids of SEQ ID NOs: 26-28, respectively; or

(3) the Va region comprises a CDR1, a CDR2, and a CDR3, comprising amino acids of SEQ ID NOs: 29-31, respectively, and the Vb region comprises a CDR1, a CDR2, and a CDR3, comprising amino acids at position 25-29, amino acids of SEQ ID NOs: 32-34, respectively.

In some embodiments, provided herein are the TCR or antigen-binding fragment that:

the Va region comprises the amino acid sequence set forth in any of SEQ ID NOs: 1, 5, or 9, or an amino acid sequence that has at least 80%, 85%, 90%, 91%, 92%, 93%, 94%, 95%, 96%, 97%, 98%, or 99% sequence identity thereto; and

the Vb region comprises the amino acid sequence set forth in any of SEQ ID NOs: 2, 6, or 10, or an amino acid sequence that has at least 80%, 85%, 90%, 91%, 92%, 93%, 94%, 95%, 96%, 97%, 98%, or 99% sequence identity thereto.

In some embodiments, provided herein are the TCR or antigen-binding fragment that the TCR or antigen-binding fragment thereof binds to or recognizes a peptide epitope of LMP2 (LLWTLVVLL) (SEQ ID NO: 16).

In some embodiments, provided herein are the TCR or antigen-binding fragment that the TCR or antigen-binding fragment thereof, when expressed on the surface of a T cell, stimulates cytotoxic activity against a target cancer cell, optionally in some embodiments, the target cancer cell contains EBV DNA sequences or expresses LMP2.

In one aspect, the disclosure is related to vector comprising a nucleic acid encoding TCR or antigen-binding fragment thereof as described herein

In some embodiments, the vector is an expression vector, a viral vector, a retroviral vector, or a lentiviral vector.

In one aspect, the disclosure is related to an engineered cell comprising the vector as described herein.

In one aspect, the disclosure is related to an engineered cell, comprising the TCR or antigen-binding fragment thereof as described herein

In some embodiments, the TCR or antigen binding fragment thereof is heterologous to the cell.

In some embodiments, the engineered cell is a cell line. In some embodiments, the engineered cell is a primary cell obtained from a subject (e.g., a human subject). In some embodiments, the engineered cell is a T cell. In some embodiments, the T cell is CD8+. In some embodiments, the T cell is CD4+.

In one aspect, the disclosure is related to a method for producing the engineered cell, comprising introducing a vector as described herein into a cell in vitro or ex vivo.

In some embodiments, the vector is a viral vector and the introducing is carried out by transduction.

In one aspect, the disclosure is related to a method of treating a disease or a disorder, comprising administering the engineered cell as described herein to a subject having a disease or disorder associated with EBV.

In some embodiments, the disease or disorder associated with EBV is a cancer.

In one aspect, the disclosure is related to a method of treating a tumor in a subject, the method includes administering to the subject in need thereof: (a) an engineered T cell, comprising: a nucleic acid encoding a TCR or antigen-binding fragment thereof that specifically binds to an antigen in a tumor; and (b) either one of both of a checkpoint inhibitor or a protein that binds to a member of the transforming growth factor beta family (TGF-β).

In one aspect, the disclosure is related to a method of treating a tumor in a subject, the method includes administering to the subject in need thereof: an engineered T cell, comprising: a nucleic acid encoding (a) a TCR or antigen-binding fragment thereof that specifically binds to an antigen in a tumor; and (b) a bifunctional trap protein that targets a checkpoint inhibitor and a member of the transforming growth factor beta family (TGF-β).

In some embodiments, the tumor is EBV-induced tumor or HPV-induced tumor.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

Exemplary embodiments are illustrated in referenced figures. It is intended that the embodiments and figures disclosed herein are to be considered illustrative rather than restrictive.

FIG. 1A is a schematic diagram showing an MP71 retroviral vector construct. P2A encodes a 2A self-cleaving peptide; Va encodes the variable region of the alpha chain of a human anti-LMP2 TCR; Vb encodes the beta chain of the human anti-LMP2 TCR; Ca encodes the constant region of the TCR alpha chain; Cb encodes the constant region of the TCR beta chain; HGH\SS and HGH\SS\2 are the signal peptides (SEQ ID NO: 14 and 15, respectively). Ψ indicates packaging sequences on viral RNA.

FIG. 1B is a schematic diagram showing an MP71 retroviral vector construct. P2A and T2A encode 2A self-cleaving peptides; Va encodes the variable region of the alpha chain of a genetically engineered human TCR; Vb encodes the beta chain of the genetically engineered human TCR; Ca encodes the constant region of the TCR alpha chain; Cb encodes the constant region of the TCR beta chain; HGH\SS and HGH\SS\2 are signal peptides (SEQ ID NO: 14 and 15, respectively); ICI-ScFv encodes the variable regions of the heavy and light chain of an immune checkpoint inhibitor (ICI) linked with a GS linker; TGFβRII encodes a ligand-binding sequence of the extracellular domain of TCRβRII; Linker is a flexible linker peptide at the C terminus of the variable heavy chain.

FIG. 2A shows the alpha chain variable domain amino acid sequence of the L201 TCR.

FIG. 2B shows the beta chain variable domain amino acid sequence of the L201 TCR.

FIG. 3A shows the DNA sequence encoding the L201 TCR α chain variable domain.

FIG. 3B shows the DNA sequence encoding the L201 TCR β chain variable domain.

FIG. 4A shows the alpha chain variable domain amino acid sequence of the L202 TCR.

FIG. 4B shows the beta chain variable domain amino acid sequence of the L202 TCR.

FIG. 5A shows the DNA sequence encoding the L202 TCR α chain variable domain.

FIG. 5B shows the DNA sequence encoding the L202 TCR β chain variable domain.

FIG. 6A shows the alpha chain variable domain amino acid sequence of the L203 TCR.

FIG. 6B shows the beta chain variable domain amino acid sequence of the L203 TCR.

FIG. 7A shows the DNA sequence encoding the L203 TCR α chain variable domain.

FIG. 7B shows the DNA sequence encoding the L203 TCR β chain variable domain.

FIG. 8 shows the amino acid sequence of HGH\SS signal peptide and the amino acid sequence of HGH\SS\2 signal peptide.

FIG. 9 is a set of graphs showing the flow cytometry results TCR expression of human T cells transduced with the constructs of L201, L202 and L203, wherein CD3, CD4 and CD8 were stained simultaneously and a viable CD3+ lymphocyte gating strategy was used. NT is a non-transduced control. TCR expression is indicated by mouse TCRβ staining.

FIG. 10 is a set of graphs showing the flow cytometry results of antigen-specific stimulated TCR-T cells, wherein the CD3, CD8 and intracellular IFN-γ were stained. L201, L202 and L203 constructs were used to transduce the cells. NT is a non-transduced control.

FIG. 11A is a graph showing the activation curve of TCR-T cells containing the anti-LMP2 TCR L201. The TCR-T cells were co-cultured with EBV peptide-pulsed APCs at 1:1 effector-to-target ratio and the percentage of T cells expressing intracellular IFN-γ (Y-axis) was measured by flow cytometry. Half maximal effective concentration (EC50) was determined.

FIG. 11B is a graph showing the activation curve of TCR-T cells containing the anti-LMP2 TCR L202. The TCR-T cells were co-cultured with EBV peptide-pulsed APCs at 1:1 effector-to-target ratio and the percentage of T cells expressing intracellular IFN-γ (Y-axis) was measured by flow cytometry. Half maximal effective concentration (EC50) was determined.

FIG. 11C is a graph showing the activation curve of TCR-T cells containing the anti-LMP2 TCR L203. The TCR-T cells were co-cultured with EBV peptide-pulsed APCs at 1:1 effector-to-target ratio and the percentage of T cells expressing intracellular IFN-γ (Y-axis) was measured by flow cytometry. Half maximal effective concentration (EC50) was determined.

FIG. 12 is a histogram showing the long-term IFN-γ production of TCR-T cells upon antigen-specific stimulation. Human T cells were transduced to express L201 TCR (TCR transduced) or untransduced (as a negative control), co-cultured with EBV peptide-pulsed APCs at 1:0, 1:1 or 3:1 effector-to-target (E:T) ratios, and the IFN-γ production was measured using a human IFN-γ ELISA kit.

FIG. 13A is a histogram showing the specific killing percentage of target cells by L201 TCR-T cells. EBV peptide-pulsed APCs were co-cultured with L201 TCR-T cells at 1:1 or 3:1 effector-to-target ratios, and the cytotoxicity of TCR-T cells were determined by measuring cell death of the APCs. Human T cells were transduced to express L201 TCR (TCR transduced) or untransduced (as a negative control).

FIG. 13B is a graph showing the relation of the specific killing percentage of target cells by L202 TCR-T cells and E:T ratios. Target and non-target cells (mixed at 1:1 ratio) were co-cultured with L202 TCR-T cells at indicated effector-to-target ratios and the cytotoxicity of TCR-T cells were determined by measuring apoptosis of target cells.

FIG. 14 is a set of graphs showing the flow cytometry results TCR expression of human T cells transduced with the constructs of E6, E6.αPD1-TGFβRII, E6.αPDL1-TGFβRII, E6.HAC-TGFβRII or E6.αgp120-TGFβRII, wherein CD3, CD4 and CD8 were stained simultaneously and a viable CD3+ lymphocyte gating strategy was used. NT is a non-transduced control. TCR expression is indicated by mouse TCRβ staining. TCR percentage is defined by the signal within the rectangular box, divided by the total signal. E6 refers to anti-E6 TCR. αPD1-TGFβRII refers to a fusion protein, wherein the extracellular domain of human TGFβRII (TGFβ Trap) is linked to the C-terminus of anti-PD-1 single chain Fv (scFV). αPDL1-TGFβRII refers to a fusion protein, wherein TGFβ Trap is linked to the C-terminus of anti-PD-L1 scFV. HAC-TGFβRII refers to a fusion protein, wherein TGFβ Trap is linked to the C-terminus of a PD-L1-binding protein named HAC. αgp120-TGFβRII refers to a fusion protein control, wherein TGFβ Trap is linked to the C-terminus of an anti-gp120 scFV.

FIG. 15A is a histogram showing the percentage of TCR-T cells expressing intracellular IFN-γ (Y-axis) upon antigen-specific stimulation. NT is a non-transduced control. TCR-T cells expressing E6, E6.αPD1-TGFβRII, E6.αPDL1-TGFβRII, E6.HAC-TGFβRII or E6.αgp120-TGFβRII TCRs were used. Peptide-pulsed A562-A2 cells were co-cultured with the TCR-T cells at 1:1 effector-to-target ratio, and the percentage of TCR-T cells expressing intracellular IFN-γ (Y-axis) was measured by flow cytometry.

FIG. 15B is a histogram showing the IFN-γ production levels of TCR-T cells transduced to express E6, E6.αPD1-TGFβRII, E6.αPDL1-TGFβRII, E6.HAC-TGFβRII or E6.αgp120-TGFβRII TCRs. NT is a non-transduced control. Ca Ski E6/E7 cells were co-cultured with the TCR-T cells at 1:0, 1:1 or 3:1 effector-to-target ratios, and the IFN-γ production in supernatant was measured using a human IFN-γ ELISA kit.

FIG. 16 is a histogram showing the specific killing percentage of target cells by TCR-T cells transduced to express E6, E6.αPD1-TGFβRII, E6.αPDL1-TGFβRII, E6.HAC-TGFβRII or E6.αgp120-TGFβRII TCRs. NT is a non-transduced control. Ca Ski tumor cells were co-cultured with TCR-T cells at 1:1 effector-to-target ratio, and the cytotoxicity of TCR-T cells were determined by measuring cell death of target cells.

FIG. 17 is a set of graphs showing the binding curves of secreted scFv-TGFβRII to TGFβ. The secreted scFv-TGFβRII was produced by 293T cells that was transduced to express E6.αPD1-TGFβRII, E6.αPDL1-TGFβRII, E6.HAC-TGFβRII or E6.αgp120-TGFβRII TCR. Binding activities were determined by ELISA.

FIG. 18 is a histogram showing the expression of TGFβ in human Ca Ski cells. CM is culture medium.

FIG. 19 is a histogram showing the proliferation of TCR-T cells upon antigen-specific stimulation. The proliferation was determined by Carboxyfluorescein succinimidyl ester (CF SE) negative population. NT is a non-transduced control. TCR-T cells were transduced to express E6.αPD1-TGFβRII, E6.αPDL1-TGFβRII, E6.HAC-TGFβRII or E6.αgp120-TGFβRII TCRs

FIG. 20 is a set of graphs showing the flow cytometry results of TCR expression in human T cells transduced with the constructs of LMP2.αPD1-TGFβRII, LMP2.αPDL1-TGFβRII, LMP2.HAC-TGFβRII or LMP2.αgp120-TGFβRII TCR.

FIG. 21 is a set of graphs showing the flow cytometry results of antigen-specific stimulated TCR-T cells, wherein CD3, CD8 and the intracellular IFN-γ were stained. L201-PD1trap (L201.αPD1-TGFβRII), L201-PDL1trap (L201.αPDL1-TGFβRII), L201-HACtrap (L201.HAC-TGFβRII), and L201-gp210trap (L201.αgp120-TGFβRII) are constructs used to transduce the cells. NT is a non-transduced control.

FIG. 22A is a histogram showing the percentage of TCR-T cells expressing intracellular IFN-γ (Y-axis) upon antigen-specific stimulation. NT is a non-transduced control. TCR-T cells expressing L201-PD1trap (L201.αPD1-TGFβRII), L201-PDL1trap (L201.αPDL1-TGFβRII), L201-HACtrap (L201.HAC-TGFβRII), or L201-gp210trap (L201.αgp120-TGFβRII) TCRs were used. Peptide-pulsed A562-A2 cells were co-cultured with the TCR-T cells at 1:1 effector-to-target ratio, and the percentage of TCR-T cells expressing intracellular IFN-γ (Y-axis) was measured by flow cytometry.

FIG. 22B is a histogram showing the IFN-γ production levels of TCR-T cells transduced to express L201-PD1trap (L201.αPD1-TGFβRII), L201-PDL1trap (L201.αPDL1-TGFβRII), L201-HACtrap (L201.HAC-TGFβRII), or L201-gp210trap (L201.αgp120-TGFβRII) TCRs. NT is a non-transduced control. Ca Ski E6/E7 cells were co-cultured with the TCR-T cells at 1:0, 1:2, 1:1 or 3:1 effector-to-target ratios, and the IFN-γ production in supernatant was measured using a human IFN-γ ELISA kit.

FIG. 23 is a graph showing the relation of the specific killing percentage of target cells by L201-trap TCR-T cells and E:T ratios. Target cells were co-cultured with TCR-T cells transduced to express L201-PD1trap (L201.αPD1-TGFβRII), L201-PDL1trap (L201.αPDL1-TGFβRII), L201-HACtrap (L201.HAC-TGFβRII), or L201-gp210trap (L201.αgp120-TGFβRII) TCRs at indicated effector-to-target (E:T) ratios and the cytotoxicity of TCR-T cells were determined by measuring cell death of target cells.

FIG. 24A is a graph showing the individual melanoma tumor volumes in mice following treatment with L202 TCR-T cells or untransduced cells.

FIG. 24B is a graph showing the average melanoma tumor volumes in mice following treatment with L202 TCR-T cells or untransduced cells.

FIG. 24C is a graph showing the tumor volume fold changes (day 20/day 0) of animals in the indicated cohorts.

FIG. 24D is a graph showing the average animal weights on the indicated days after L202 TCR-T cell or untransduced cell administration.

FIG. 25 shows the CDR sequences for three T cell receptors.

FIG. 26 provides sequences that are described in the present disclosure.

### DETAILED DESCRIPTION OF INVENTION

The following embodiments and aspects thereof are described and illustrated in conjunction with systems, compositions, and methods which are meant to be exemplary and illustrative, not limiting in scope.

### Definitions

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

As used herein, the term “about” refers to a measurable value such as an amount, a time duration, and the like, and encompasses variations of ±20%, ±10%, ±5%, ±1%, ±0.5% or ±0.1% from the specified value.

As used herein, the term “antibody” refers to an intact immunoglobulin or to a monoclonal or polyclonal antigen-binding fragment with the Fc (crystallizable fragment) region or FcRn binding fragment of the Fc region, referred to herein as the “Fc fragment” or “Fc domain”. Antigen-binding fragments may be produced by recombinant DNA techniques or by enzymatic or chemical cleavage of intact antibodies. Antigen-binding fragments include, inter alia, Fab, Fab′, F(ab′)2, Fv, dAb, and complementarity determining region (CDR) fragments, single-chain antibodies (scFv), single domain antibodies, chimeric antibodies, diabodies and polypeptides that contain at least a portion of an immunoglobulin that is sufficient to confer specific antigen binding to the polypeptide. The Fc domain includes portions of two heavy chains contributing to two or three classes of the antibody. The Fc domain may be produced by recombinant DNA techniques or by enzymatic (e.g. papain cleavage) or via chemical cleavage of intact antibodies.

The term “antibody fragment,” as used herein, refers to a protein fragment that comprises only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 341, 544-546 (1989)) which consists of a VH domain; (vii) isolated CDR regions; (viii) F(ab′)2 fragments, a bivalent fragment including two Fab′ fragments linked by a disulphide bridge at the hinge region; (ix) single chain antibody molecules (e.g., single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (VH-CH1-VH-CH1) which, together with complementary light chain polypeptides, form a pair of antigen binding regions (Zapata et al. Protein Eng. 8(10):1057-1062 (1995); and U.S. Pat. No. 5,641,870).

“Single chain variable fragment”, “single-chain antibody variable fragments” or “scFv” antibodies as used herein refers to forms of antibodies comprising the variable regions of only the heavy (VH) and light (VL) chains, connected by a linker peptide. The scFvs are capable of being expressed as a single chain polypeptide. The scFvs retain the specificity of the intact antibody from which it is derived. The light and heavy chains may be in any order, for example, VH-linker-VL or VL-linker-VH, so long as the specificity of the scFv to the target antigen is retained.

The term “binding protein” refers to natural protein binding domains (such as cytokine, cytokine receptors), antibody fragments (such as Fab, scFv, diabody, variable domain derived binders, VHH nanobody), alternative scaffold derived protein binding domains (such as Fn3 variants, ankyrin repeat variants, centyrin variants, avimers, affibody) or any protein recognizing specific antigens.

As used herein, the term “antigen” refers to a molecule capable of being bound by an antibody or a T cell receptor (TCR) if presented by MHC molecules. The term “antigen”, as used herein, also encompasses T-cell epitopes which are recognized by T-cell receptors. This recognition causes activation of T-cells and subsequent effector mechanisms such as proliferation of the T cells, cytokine secretion, etc. An antigen is additionally capable of being recognized by the immune system and/or capable of inducing a humoral immune response and/or a cellular immune response leading to the activation of B lymphocytes and/or T lymphocytes.

As used herein, the term “HPV antigen” refers to a polypeptide molecule derived from Human Papilloma Virus (HPV), preferably wherein the HPV is selected from HPV1, HPV2, HPV3, HPV4, HPV6, HPV10, HPV11, HPV16, HPV18, HPV26, HPV27, HPV28, HPV29, HPV30, HPV31, HPV33, HPV34, HPV35, HPV39, HPV40, HPV41, HPV42, HPV43, HPV45, HPV49, HPV51, HPV52, HPV54, HPV55, HPV56, HPV57, HPV58, HPV59, HPV68, HPV69. More preferably, the HPV is selected from high risk HPVs, for example, HPV16, HPV18, HPV31, HPV33, HPV35, HPV39, HPV45, HPV51, HPV52, HPV56, HPV58, HPV59, HPV68, HPV69. In some embodiments, the HPV polypeptide molecule is selected from E6 and E7.

As used herein, the term “EBV antigen” refers to a polypeptide molecule derived from Epstein-Barr virus (EBV). EBV antigen includes, but is not limited to, the latent membrane proteins (LMP1, LMP2A, and LMP2B) and the Epstein-Barr nuclear antigens (EBNA1, -2, -3A, -3B, -3C, -LP).

As used herein, the term “peripheral blood cell subtypes” refers to cell types normally found in the peripheral blood including, but not limited to, eosinophils, neutrophils, T cells, monocytes, K cells, granulocytes, and B cells.

As used herein, the term “T cell” includes CD4+ T cells and CD8+ T cells. The term T cell also includes both T helper 1 type T cells and T helper 2 type T cells. T cells express a cell surface receptor that recognizes a specific antigenic moiety on the surface of a target cell. The cell surface receptor may be a wild type or recombinant T cell receptor (TCR), a chimeric antigen receptor (CAR), or any other surface receptor capable of recognizing an antigenic moiety that is associated with the target cell. Typically, a TCR has two protein chains (alpha- and beta-chain), which bind to specific peptides presented by an MHC protein on the surface of certain cells. TCRs recognize peptides in the context of MHC molecules expressed on the surface of a target cell. TCRs also recognize cancer antigens presented directly on the surface of cancer cells.

“Genetically modified cells”, “redirected cells”, “engineered cells”, “genetically engineered cells” or “modified cells” as used herein refer to cells that express the genetically engineered antigen receptors and checkpoint inhibitors. In some embodiments, the genetically modified cells comprise vectors that encode a genetically engineered TCR and vectors that encode one or more checkpoint inhibitors. In some embodiments, the genetically modified cells comprise a vector that encodes a genetically engineered TCR and one or more checkpoint inhibitors. In one embodiment, the genetically modified cell is a T lymphocyte (T cell). In one embodiment, the genetically modified cell is a Natural Killer (NK) cell.

As used herein, the term “genetically engineered” or “genetically modified” refers to a modification of a nucleic acid sequence of a cell, including, but not limited to, deleting a coding or non-coding region or a portion thereof or inserting a coding region or a portion thereof.

As used herein, the term “vector”, “cloning vector” or “expression vector” refers to a vehicle by which a polynucleotide sequence (e.g. a foreign gene) can be introduced into a host cell, so as to transform the host and promote expression (e.g. transcription and translation) of the introduced sequence. Vectors include plasmids, phages, viruses, etc. Most popular type of vector is a “plasmid”, which refers to a closed circular double stranded DNA loop into which additional DNA segments comprising gene of interest may be ligated. Another type of vector is a viral vector, in which a nucleic acid construct to be transported is ligated into the viral genome. Viral vectors are capable of autonomous replication in a host cell into which they are introduced or may integrate themselves into the genome of a host cell and thereby are replicated along with the host genome. Moreover, certain vectors are capable of directing the expression of genes to which they are operatively linked. Such vectors are referred to herein as “recombinant expression vectors” or simply “expression vectors”. It may be noted that the invention is intended to include such other forms of expression vectors, such as viral vectors (e.g., replication defective retroviruses, adenoviruses and adeno-associated viruses), which serve equivalent functions.

As used herein, the term “retroviral vector” or “recombinant retroviral vector” refers to a nucleic acid construct which carries, and within certain embodiments, is capable of directing the expression of a nucleic acid molecule of interest. A retrovirus is present in the RNA form in its viral capsule and forms a double-stranded DNA intermediate when it replicates in the host cell. Similarly, retroviral vectors are present in both RNA and double-stranded DNA forms, both of which forms are included in the term “retroviral vector” and “recombinant retroviral vector”. The term “retroviral vector” and “recombinant retroviral vector” also encompass the DNA form which contains a recombinant DNA fragment and the RNA form containing a recombinant RNA fragment. The vectors may include at least one transcriptional promoter/enhancer, or other elements which control gene expression. Such vectors may also include a packaging signal, long terminal repeats (LTRs) or portion thereof, and positive and negative strand primer binding sites appropriate to the retrovirus used (if these are not already present in the retroviral vector). Optionally, the vectors may also include a signal which directs polyadenylation, selectable markers such as Ampicillin resistance, Neomycin resistance, TK, hygromycin resistance, phleomycin resistance histidinol resistance, or DHFR, as well as one or more restriction sites and a translation termination sequence. By way of example, such vectors may include a 5′ LTR, a leading sequence, a tRNA binding site, a packaging signal, an origin of second strand DNA synthesis, and a 3′ LTR or a portion thereof.

“Linker” (L) or “linker domain” or “linker region” as used herein refers to an oligo- or polypeptide region from about 1 to 100 amino acids in length, which links together any of the domains/regions of the TCR of the invention. Linkers may be composed of flexible residues like glycine and serine so that the adjacent protein domains are free to move relative to one another. Longer linkers may be used when it is desirable to ensure that two adjacent domains do not sterically interfere with one another. Linkers may be cleavable or non-cleavable. Examples of cleavable linkers include 2A linkers (for example T2A), 2A-like linkers or functional equivalents thereof and combinations thereof. In some embodiments, the linkers include the picornaviral 2A-like linker, CHYSEL sequences of porcine teschovirus (P2A), Thosea asigna virus (T2A) or combinations, variants and functional equivalents thereof. In other embodiments, the linker sequences may comprise Asp-Val/Ile-Glu-X-Asn-Pro-Gly(2A)-Pro(2B) motif, which results in cleavage between the 2A glycine and the 2B proline. Other linkers will be apparent to those of skill in the art and may be used in connection with alternate embodiments of the invention.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, a “subject” is a mammal, such as a human or other animal, and typically is human. In some embodiments, the subject, e.g., patient, to whom the cells, cell populations, or compositions are administered is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

The term “control” refers to any reference standard suitable to provide a comparison to the expression products in the test sample.

As used herein, the term “inhibit” refers to any decrease in, for example a particular action, function, or interaction. For example, a biological function, such as the function of a protein and/or binding of one protein to another, is inhibited if it is decreased as compared to a reference state, such as a control like a wild-type state or a state in the absence of an applied agent. For example, the binding of a PD-1 protein to one or more of its ligands, such as PD-L1 and/or PD-L2, and/or resulting PD-1 signaling and immune effects is inhibited or deficient if the binding, signaling, and other immune effects are decreased due to contact with an agent, such as an anti-PD-1 antibody, in comparison to when the PD-1 protein is not contacted with the agent. Such inhibition or deficiency can be induced, such as by application of agent at a particular time and/or place, or can be constitutive, such as by continual administration. Such inhibition or deficiency can also be partial or complete (e.g., essentially no measurable activity in comparison to a reference state, such as a control like a wild-type state). Essentially complete inhibition or deficiency is referred to as blocked.

“Conditions” and “disease conditions,” as used herein may include, cancers, tumors or infectious diseases. In exemplary embodiments, the conditions include but are in no way limited to any form of malignant neoplastic cell proliferative disorders or diseases. In exemplary embodiments, conditions include any one or more of kidney cancer, melanoma, prostate cancer, breast cancer, glioblastoma, lung cancer, colon cancer, or bladder cancer.

“Cancer” and “cancerous” refers to or describe the physiological condition in mammals that is typically characterized by unregulated cell growth. The term “cancer” is meant to include all types of cancerous growths or oncogenic processes, metastatic tissues or malignantly transformed cells, tissues, or organs, irrespective of histopathologic type or stage of invasiveness. Examples of solid tumors include malignancies, e.g., sarcomas, adenocarcinomas, and carcinomas, of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. In one embodiment, the cancer is a melanoma, e.g., an advanced stage melanoma. Metastatic lesions of the aforementioned cancers can also be treated or prevented using the methods and compositions of the invention. Examples of other cancers that can be treated include bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin Disease, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of the cancers. Treatment of metastatic cancers, e.g., metastatic cancers that express PD-L1 (Iwai et al. (2005) Int. Immunol. 17:133-144) can be effected using the antibody molecules described herein.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder, such as cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment). In some embodiments, treatment of cancer includes decreasing tumor volume, decreasing the number of cancer cells, inhibiting cancer metastases, increasing life expectancy, decreasing cancer cell proliferation, decreasing cancer cell survival, or amelioration of various physiological symptoms associated with the cancerous condition.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount. In the context of lower tumor burden, the prophylactically effective amount in some aspects will be higher than the therapeutically effective amount.

In accordance with various embodiments described herein, the present invention provides engineered cells and compositions/formulations containing the engineered cells. The present invention also provides methods or processes for manufacturing the engineered cells, which may be useful for treating patients with a pathological disease or condition.

Further, in accordance with various embodiments described herein, the present invention provides a recombinant vector comprising a nucleic acid construct suitable for genetically modifying a cell, which may be used for treatment of pathological disease or condition.

Furthermore, in accordance with various embodiments described herein, the present invention provides an engineered cell comprising a nucleic acid construct suitable for genetically modifying a cell, which may be used for treatment of pathological disease or condition, wherein the nucleic acid encodes: (a) a genetically engineered antigen receptor that specifically binds to an antigen; and (b) an inhibitory protein that reduces, or is capable of effecting reduction of, expression of a tumor target. In various embodiments, the cell expresses the genetically engineered antigen receptor and the inhibitory protein. In various embodiments, the inhibitory protein is constitutively expressed.

Among the diseases, conditions, and disorders for treatment with the provided cells, compositions, methods and uses are tumors, including solid tumors, hematologic malignancies, and melanomas, and infectious diseases, such as infection with a virus or other pathogen, e.g., HPV, HIV, HCV, HBV, EBV, HTLV-1, CMV, adenovirus, BK polyomarvirus, HHV-8, MCV or other pathogens, and parasitic disease. In some embodiments, the disease or condition is a tumor, cancer, malignancy, neoplasm, or other proliferative disease or disorder. Such diseases include but are not limited to leukemia, lymphoma, e.g., chronic lymphocytic leukemia (CLL), acute-lymphoblastic leukemia (ALL), non-Hodgkin's lymphoma, acute myeloid leukemia, multiple myeloma, refractory follicular lymphoma, mantle cell lymphoma, indolent B cell lymphoma, B cell malignancies, cancers of the uterine cervix, colon, lung, liver, breast, prostate, ovarian, skin, melanoma, bone, and brain cancer, ovarian cancer, epithelial cancers, renal cell carcinoma, pancreatic adenocarcinoma, Hodgkin lymphoma, cervical carcinoma, colorectal cancer, glioblastoma, neuroblastoma, Ewing sarcoma, medulloblastoma, osteosarcoma, synovial sarcoma, and/or mesothelioma.

### T Cell Receptors and Binding Molecules

The present disclosure provides a T cell receptor (TCR) or antigen-binding fragment thereof. In some embodiments, a “T cell receptor” or “TCR” is a molecule that contains a variable a (or alpha) and b (or beta) chains (also known as TCRα and TCRβ, respectively) or a variable g (or gamma) and d (or delta) chains (also known as TCRγ and TCRδ, respectively), or antigen-binding portions thereof, and which is capable of specifically binding to an antigen, e.g., a peptide antigen or peptide epitope bound to an MHC molecule. In some embodiments, the TCR is in the ab form. Typically, TCRs that exist in aβ and γδ forms are generally structurally similar, but T cells expressing them may have distinct anatomical locations or functions. Generally, a TCR is found on the surface of T cells (or T lymphocytes) where it is generally responsible for recognizing antigens, such as peptides bound to major histocompatibility complex (MHC) molecules.

In some embodiments, the TCR is an intact or full-length TCR, such as a TCR containing the a chain and b chain. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable a (Va) chain and variable b (Vb) chain of a TCR, or antigen-binding fragments thereof sufficient to form a binding site for binding to a specific MHC-peptide complex.

The variable domains of the TCR contain complementarity determining regions (CDRs), which generally are the primary contributors to antigen recognition and binding capabilities and specificity of the peptide, MHC and/or MHC-peptide complex. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores el al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex.

In some embodiments, the a-chain and/or b-chain of a TCR also can contain a constant domain, a transmembrane domain and/or a short cytoplasmic tail (see, e.g., Janeway et al., Immunobiology: The Immune System in Health and Disease, 3 Ed., Current Biology Publications, p. 4:33, 1997). In some aspects, each chain (e.g. alpha or beta) of the TCR can possess one N-terminal immunoglobulin variable domain, one immunoglobulin constant domain, a transmembrane region, and a short cytoplasmic tail at the C-terminal end. In some embodiments, a TCR, for example via the cytoplasmic tail, is associated with invariant proteins of the CD3 complex involved in mediating signal transduction. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3y, CD35, CD3e and CD3z chains) contain one or more immunoreceptor tyrosine-based activation motif or IT AM and generally are involved in the signaling capacity of the TCR complex.

It is within the level of a skilled artisan to determine or identify the various domains or regions of a TCR. In some cases, the exact locus of a domain or region can vary depending on the particular structural or homology modeling or other features used to describe a particular domain. It is understood that reference to amino acids, including to a specific sequence set forth as a SEQ ID NO used to describe domain organization of a TCR are for illustrative purposes and are not meant to limit the scope of the embodiments provided. In some cases, the specific domain (e.g. variable or constant) can be several amino acids (such as one, two, three or four) longer or shorter. In some aspects, residues of a TCR are known or can be identified according to the International Immunogenetics Information System (IMGT) numbering system (see e.g. www.imgt.org; see also, Lefranc et al. (2003) Developmental and Comparative Immunology, 27(1); 55-77; and The T Cell Factsbook 2nd Edition, Lefranc and LeFranc Academic Press 2001).

In some embodiments, the a chain and b chain of a TCR each further contain a constant domain. In some embodiments, the a chain constant domain (Ca) and b chain constant domain (Cb) individually are mammalian, such as is a human or murine constant domain. In some embodiments, the constant domain is adjacent to the cell membrane. For example, in some cases, the extracellular portion of the TCR formed by the two chains contains two membrane-proximal constant domains, and two membrane-distal variable domains, which variable domains each contain CDRs.

In some aspects, provided herein are TCRs that contains a human constant region, such as an alpha chain containing a human Ca region and a beta chain containing a human Cb. In some embodiments, the provided TCRs are fully human. Among the provided TCRs are TCRs containing a human constant region, such as fully human TCRs, whose expression and/or activity, such as when expressed in human cells, e.g. human T cells, such as primary human T cells, are not impacted by or are not substantially impacted by the presence of an endogenous human TCR.

In some embodiments, the engineered TCRs are expressed at similar or improved levels on the cell surface, exhibit the similar or greater functional activity (e.g. cytolytic activity) and/or exhibit similar or greater anti-tumor activity, when expressed by human cells that contain or express an endogenous human TCR, such as human T cells, as compared to the level of expression, function activity and/or anti-tumor activity of the same TCR in similar human cells but in which expression of the endogenous TCR has been reduced or eliminated. In some examples an engineered TCR as described herein herein, when expressed in human T cells, is expressed on the cell surface at a level that is at least or at least about 80%, 85%, 90%, 95%, 100%, 105%, 110%, 115% or 120% of the level of expression of the same TCR when expressed in similar human T cells but in which expression of the endogenous TCR has been reduced or eliminated.

In some embodiments, each of the Ca and Cb domains is human. In some embodiments, the Ca is encoded by the TRAC gene (IMGT nomenclature) or is a variant thereof. In some embodiments, the variant of a Ca contains replacement of at least one non native cysteine, such as any replacement described herein.

In some embodiments, the TCR may be a heterodimer of two chains a and b that are linked, such as by a disulfide bond or disulfide bonds. In some embodiments, the constant domain of the TCR may contain short connecting sequences in which a cysteine residue forms a disulfide bond, thereby linking the two chains of the TCR. In some embodiments, a TCR may have an additional cysteine residue in each of the a and b chains, such that the TCR contains two disulfide bonds in the constant domains. In some embodiments, each of the constant and variable domains contains disulfide bonds formed by cysteine residues.

In some embodiments, the TCR comprises CDRs, Va and/or Vb and constant region sequences as described herein.

In some embodiments, the TCR is a full-length TCR. In some embodiments, the TCR is an antigen-binding portion. In some embodiments, the TCR is a dimeric TCR (dTCR). In some embodiments a dTCR contains a first polypeptide wherein a sequence corresponding to a provided TCR α chain variable region sequence is fused to the N terminus of a sequence corresponding to a TCR α chain constant region extracellular sequence, and a second polypeptide wherein a sequence corresponding to a provided TCR b chain variable region sequence is fused to the N terminus a sequence corresponding to a TCR b chain constant region extracellular sequence, the first and second polypeptides being linked by a disulfide bond.

In some embodiments, a TCR may be cell-bound or in soluble form. In some embodiments, the TCR is in cell-bound form expressed on the surface of a cell.

In some embodiments, the TCR is a single chain TCR (scTCR). The scTCR is a single amino acid strand containing an a chain and a b chain that is able to bind to MHC-peptide complexes. Typically, a scTCR can be generated using methods known to those of skill in the art, See e.g., WO 96/13593, WO 96/18105, WO99/18129, WO 04/033685, WO2006/037960, WO2011/044186; U.S. Pat. No. 7,569,664; each of which is incorporated herein by reference in its entirety.

Provided herein are binding molecules, such as those that bind or recognize a peptide epitope associated with an antigen (e.g., a cancer antigen). In some embodiments, the antigen can be a peptide epitope expressed on the surface of a cancer cell and/or a cell infected with Epstein-Barr virus (EBV) or human papillomavirus (HPV), in the context of an MHC molecule. Such binding molecules include T cell receptors (TCRs) and antigen-binding fragments thereof and antibodies and antigen binding fragments thereof that exhibit antigenic specificity for binding or recognizing such peptide epitopes. Also provided in some embodiments are nucleic acid molecules encoding the binding molecules, engineered cells containing the binding molecules, compositions and methods of treatment involving administering such binding molecules, engineered cells or compositions. In some aspects, engineered cells that express a provided binding molecule, e.g. a TCR or antigen-binding fragment, exhibit cytotoxic activity against target cells expressing the peptide epitope, such as cancer cells or cells that are infected with EBV.

In some aspects, this disclosure provides binding molecules, including a TCR or antigen binding fragment thereof or an antibody, e.g., antibody fragments thereof, and proteins such as chimeric molecules containing one or more of the foregoing, such as the chimeric receptors, e.g., TCR-like CARs, and/or engineered cells expressing the TCRs or CARs, bind to a peptide epitope derived from EBV. In some embodiments, the binding molecule is an anti-LMP2 binding molecule.

In some aspects, the binding molecule recognizes or binds epitopes in the context of an MHC molecule, such as an MHC Class I molecule. In some aspects, the MHC Class I molecule is a human leukocyte antigen (HLA)-A2 molecule, including any one or more subtypes thereof, e.g. HLA-A*0201, *0202, *0203, *0206, or *0207. In some cases, there can be differences in the frequency of subtypes between different populations. For example, in some embodiments, more than 95% of the HLA-A2 positive Caucasian population is HLA-A*0201, whereas in the Chinese population the frequency has been reported to be approximately 23% HLA-A*0201, 45% HLA-A*0207, 8% HLA-A*0206 and 23% HLA-A*0203. In some embodiments, the MEW molecule is HLA-A*0201. In some embodiments, the present disclosure provides TCR or antigen-binding fragment thereof that bind an EBV-LMP2/HLA-A02 complex.

In some embodiments, the binding molecule, e.g., TCR or antigen-binding fragment thereof or antibody or antigen-binding fragment thereof, is isolated or purified or is recombinant. In particular embodiments, any of the provided binding molecules, e.g. TCRs or antigen-binding fragments thereof or antibody or antigen-binding fragments thereof, are recombinant. In some aspects, the binding molecule, e.g., TCR or antigen-binding fragment thereof or antibody or antigen-binding fragment thereof, is human. In some embodiments, the binding molecule is monoclonal. In some aspects, the binding molecule is a single chain. In other embodiments, the binding molecule contains two chains. In some embodiments, the binding molecule, e.g., TCR or antigen-binding fragment thereof or antibody or antigen-binding fragment thereof, is expressed on the surface of a cell.

In some embodiments, the Va region comprises the amino acid sequence set forth in any of SEQ ID NOs: 1, 5, or 9, or an amino acid sequence that has at least 80%, 85%, 90%, 91%, 92%, 93%, 94%, 95%, 96%, 97%, 98%, or 99% sequence identity thereto. In some embodiments, the Vb region comprises the amino acid sequence set forth in any of SEQ ID NOs: 2, 6, or 10, or an amino acid sequence that has at least 80%, 85%, 90%, 91%, 92%, 93%, 94%, 95%, 96%, 97%, 98%, or 99% sequence identity thereto. In some embodiments, the Va region comprises one or more Va CDR sequences as described herein. In some embodiments, the Vb region comprises one or more Vb CDR sequences as described herein.

The present disclosure also provides TCR a and/or b chain as described herein. In some embodiments, the a chain comprises the amino acid sequence set forth in any of SEQ ID NOs: 35, 37, or 39, or an amino acid sequence that has at least 80%, 85%, 90%, 91%, 92%, 93%, 94%, 95%, 96%, 97%, 98%, or 99% sequence identity thereto. In some embodiments, the b chain comprises the amino acid sequence set forth in any of SEQ ID NOs: 36, 38, or 40, or an amino acid sequence that has at least 80%, 85%, 90%, 91%, 92%, 93%, 94%, 95%, 96%, 97%, 98%, or 99% sequence identity thereto. In some embodiments, the a chain comprises one or more Va CDR sequences as described herein. In some embodiments, the b chain comprises one or more Vb CDR sequences as described herein.

### Epstein-Barr Virus Infection and Cancer

Epstein Barr Virus (EBV) was one of the first viruses to be identified as oncogenic. EBV is extremely effective in infecting B cells through its interaction with CD21 and MHC class II. EBV can also infect and be retained in epithelial cells. Virtually all adults in the world have been exposed to EBV. In the absence of immune compromise, initial exposure in childhood results in a self-limited illness controlled by a cellular immune response. The presence of an immune defense against Epstein Barr Virus (EBV) and EBV-associated disease is well known. The host's generation of antigen specific T-cells against viral proteins is very effective against the virus. However, EBV can persist in epithelial or B cells without being completely eliminated. Any changes in the immune status of the host can lead to re-activation and depending on the degree of immune compromise, this re-activation can lead to malignancy.

EBV is involved in solid organ and hematopoietic cell transplantation (HSCT) where the decreased number or absence of T-cells may cause un-restricted proliferation of B-cells harboring EBV. Such uncontrolled expansion can lead to post transplant lymphoproliferative disease (PTLD), the most common post-transplant malignancy. The frequency and intensity of this syndrome varies within each patient and the effects of their immune suppression on their T-cell population. EBV is also involved in other malignancies. Several lines of research have implicated EBV in the pathogenesis of various epithelial and lymphoid malignancies. For example, it is well known that Hodgkin (Glaser, et al. “Epstein-Barr virus-associated Hodgkin's disease: epidemiologic characteristics in international data.” International journal of cancer 70.4 (1997): 375-382) and non-Hodgkin Lymphomas are related to EBV. There is also a clear causal relationship between EBV and nasopharyngeal carcinoma (NPC; Raab-Traub “Nasopharyngeal carcinoma: an evolving role for the Epstein-Barr virus.” Epstein Barr Virus Volume 1. Springer, Cham, 2015. 339-363). Tumor samples of patients with Hodgkin Lymphoma and NPC express EBV derived proteins including the latent membrane protein 2 (LMP2). LMP-2 has also been found in 40% of EBV-related gastric carcinoma. Because these are non-self and are also the main targets of the cellular immune response against EBV, these represent ideal targets for immunotherapy approaches.

The list of LMP2(+) human malignancies associated with EBV includes Burkitt's lymphoma, immunosuppressive lymphoma, diffuse large B-cell lymphoma, diffuse large B-cell lymphoma associated with chronic inflammation, lymphomatoid granulomatosis, plasmablastic lymphoma, primary effusion lymphoma, post-transplant lymphoproliferative disorder, nasopharyngeal carcinoma, gastric adenocarcinoma, lymphoepithelioma-associated carcinoma, and immunodeficiency-related leiomyosarcoma. These disorders are described e.g., in WO/2019/213416 A1; Thompson et al., “Epstein-Barr virus and cancer.” Clinical Cancer Research 10.3 (2004): 803-821, both of which are incorporated herein by reference in the entirety.

The EBV infection/transformation of resting B-cells produces Latent Lymphoblastoma Lines (LCL). LCLs present in latent replication and carry multiple copies of the viral genome as an episome. They express a number of viral gene products denominated latent proteins that vary according to latency stage. A total of ten latency proteins have been described: Six Epstein Virus Nuclear Antigens (EBNA 1, 2, 3A, 3B, 3C and LP), three Latent Membrane Proteins (LMP 1, 2A and 2B) and BARF1. Initial EBV infection activates B-cells and induces latency III when EBNA1, EBNA2, EBNA3, LMP1, LMP2 and BARF1 are expressed. These proteins are described e.g., in Bollard, et al., “T-cell therapy in the treatment of post-transplant lymphoproliferative disease.” Nature reviews Clinical oncology 9.9 (2012): 510, which is incorporated herein by reference in its entirety.

The present disclosure provides methods of treating EBV infection and/or EBV induced disease and disorders.

### Engineered Cells

The present disclosure provides engineered cells (e.g., T cells) that comprise TCR or antigen-binding fragment thereof, or other similar antigen-binding molecules as described herein. These engineered cells can be used to treat various disorders or disease as described herein (e.g., virus infection, cancers, virus-induced disorders).

In various embodiments, the cell that is engineered is obtained from including but are not limited from animal and humans. In various embodiments, the cell that is engineered is hemocyte including but is not limited to leukocyte, lymphocyte or any other suitable blood cell type. Preferably, the cell is a peripheral blood cell. More preferably, the cell is a T cell, B cell or NK cell.

In another embodiments, the cell is a T cell. Examples of the T cell used in the present invention include, but are not limited to: cell obtained by in vitro culture of T cells (e.g., tumor infiltrating lymphocytes) isolated from patient(s); TCR gene-modified T cells obtained by transducing T cells, isolated from the peripheral blood of patient(s), with a viral vector; and CAR-transduced T cells. Preferably, the T cell is a TCR gene-modified T cell.

In an embodiment of the invention, the cell is an NK cell.

In some embodiments, preparation of the engineered cells includes one or more culture and/or preparation steps. The cells for introduction of the binding molecule, e.g., TCR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

Also provided are methods, nucleic acids, compositions, and kits, for expressing the binding molecules, and for producing the genetically engineered cells expressing such binding molecules. The genetic engineering generally involves introduction of a nucleic acid encoding the therapeutic molecule, e.g. TCR, CAR, e.g. TCR-like CAR, polypeptides, fusion proteins, into the cell, such as by retroviral transduction, transfection, or transformation. In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical application.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors. In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), or spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In some embodiments, the vector is a lentivirus vector. In some embodiments, recombinant nucleic acids are transferred into T cells via electroporation. In some embodiments, recombinant nucleic acids are transferred into T cells via transposition. Other methods of introducing and expressing genetic material in immune cells include calcium phosphate transfection, protoplast fusion, cationic liposome-mediated transfection; tungsten particle-facilitated microparticle bombardment and strontium phosphate DNA co-precipitation. Many of these methods are descried e.g., in WO2019195486, which is incorporated herein by reference in its entirety.

### Recombinant Vectors

Any vector or vector type may be used to deliver genetic material to the cell. These vectors include but are not limited to plasmid vectors, viral vectors, BACs, YACs, and HACs. Accordingly, viral vectors may include but are not limited to recombinant retroviral vectors, recombinant lentiviral vectors, recombinant adenoviral vectors, foamy virus vectors, recombinant adeno-associated viral (AAV) vectors, hybrid vectors, and plasmid transposons (for example sleeping beauty transposon system) or integrase based vector systems. Other vectors that may be used in connection with alternate embodiments of the invention will be apparent to those of skill in the art.

In another embodiments, the vector used is a recombinant retroviral vector. The viral vector may be grown in a culture medium specific for viral vector manufacturing. Any suitable growth media and/or supplements for growing viral vectors may be used in accordance with the embodiments described herein.

### Genetically Engineered Antigen Receptors

The antigen receptor that is genetically engineered includes but is not limited to T cell receptors (TCRs), Killer-cell immunoglobulin-like receptor family (KIRs), C-type lectin receptor family, Leukocyte immunoglobulin-like receptor family (LILRs), Type 1 cytokine receptors, Type 2 cytokine receptor family, Tumor necrosis factor family, TGFβ receptor family, chemokine receptors, and IgSF.

In an embodiment of the invention, the genetically engineered antigen receptor encoded by the nucleic acid construct comprises a genetically engineered NK cell receptor. In some embodiments, the NK cell receptor comprises Killer-cell immunoglobulin-like receptor family (KIRs). In alternate embodiments, the NK cell receptor comprises C-type lectin receptor family.

In other embodiments, the genetically engineered antigen receptor encoded by the nucleic acid construct comprises a genetically engineered T cell receptor (TCR). In one embodiment, the T cell expressing TCR is an aβ-T cell. In alternate embodiments, the T cell expressing TCR is a γδ-T cell.

### Antigens Targeted

In some embodiments, the antigen associated with the disease or disorder is selected from the group consisting of molecules expressed by HPV, HIV, HCV, HBV, EBV, HTLV-1, CMV, adenovirus, BK polyomarvirus, MCV or other pathogens, orphan tyrosine kinase receptor ROR1, tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethycholine e receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, CS-1, c-Met, GD-2, and MAGE A3 and/or biotinylated molecules.

The genetically engineered antigen receptor binds to antigens from Human papillomavirus (HPV). The sub-type of HPV is selected from but not limited to, HPV1, HPV2, HPV3, HPV4, HPV6, HPV10, HPV11, HPV16, HPV18, HPV26, HPV27, HPV28, HPV29, HPV30, HPV31, HPV33, HPV34, HPV35, HPV39, HPV40, HPV41, HPV42, HPV43, HPV45, HPV49, HPV51, HPV52, HPV54, HPV55, HPV56, HPV57, HPV58, HPV59, HPV68, HPV69. In some embodiments, the sub-type of HPV targeted by the genetically engineered antigen receptor is selected from at least one high-risk HPV, for example but not limited to HPV16, HPV18, HPV31, HPV33, HPV35, HPV39, HPV45, HPV51, HPV52, HPV56, HPV58, HPV59, HPV68, HPV69.

In some embodiments, the HPV antigen includes but is not limited to, E1, E2, E3, E4, E6 and E7, L1 and L2 proteins. In another embodiments, the antigen is an E6 antigen. In yet another embodiment, the antigen is an E7 antigen. In another embodiment, the antigen is an HPV16 E6 antigen.

In other embodiments, the genetically engineered antigen receptor binds to antigens from EBV. The EBV antigen is selected from but not limited to the latent membrane proteins (LMP1, LMP2A, LMP2B) and the Epstein-Barr nuclear antigens (EBNA1, -2, -3A, -3B, -3C, -LP).

Accordingly, the disease or condition treated is an infectious disease or condition, such as, but not limited to, viral, retroviral, bacterial, and protozoal infections, immunodeficiency, Human Papilloma Virus (HPV), Cytomegalovirus (CMV), Epstein-Barr virus (EBV), adenovirus, BK polyomavirus. In some embodiments, the disease or condition is a virus associated malignancy for example, but not limited to, HPV, HCV, EBV, HIV, HHV-8, HTLV-1, and MCV. Preferably, the viral-associated malignancy for treatment with the provided compositions, cells, methods and uses is an HPV or EBV associated cancer. Moreover, the provided compositions, cells, and methods can be used for the treatment of solid tumors caused by an HPV or EBV associated cancer. Specifically, the diseases or conditions include HPV associated cancers include, but are not limited to, cancer of uterine cervix, oropharynx, anus, anal canal, anorectum, vagina, vulva, and penis. The diseases or conditions include HPV associated head and neck cancers, HPV associated cancer of uterine cervix. Specifically, the diseases or conditions also include EBV associated cancers, for example, nasopharyngeal cancer, lymphomas, breast cancer and hepatocellular carcinoma.

### Checkpoint Inhibitors

In various embodiments, the engineered cell expresses at least one checkpoint inhibitor (CPI). The inhibitory protein or CPI expressed by the engineered cells of the present invention inhibits or blocks an immune checkpoint, wherein the immune checkpoints comprises PD-1, PD-L1, PD-L2, 2B4 (CD244), 4-1BB, A2aR, B7.1, B7.2, B7-H2, B7-H3, B7-H4, B7-H6, BTLA, butyrophilins, CD160, CD48, CTLA4, GITR, gp49B, HHLA2, HVEM, ICOS, ILT-2, ILT-4, KIR family receptors, LAG-3, OX-40, PIR-B, SIRPalpha (CD47), TFM-4, TIGIT, TIM-1, TIM-3, TIM-4, VISTA and combinations thereof.

In some embodiments, the inhibitory protein blocks PD-1 or PD-L1. In various embodiments, the inhibitory protein comprises an anti-PD-1 scFv. The inhibitory protein is capable of leading to reduced expression of PD-1 or PD-L1 and/or inhibiting upregulation of PD-1 or PD-L1 in T cells in the population and/or physically obstructing the formation of the PD-1/PD-L1 complex and subsequent signal transduction. In one embodiment, the inhibitory protein blocks PD-1.

### Nucleic Acid Constructs

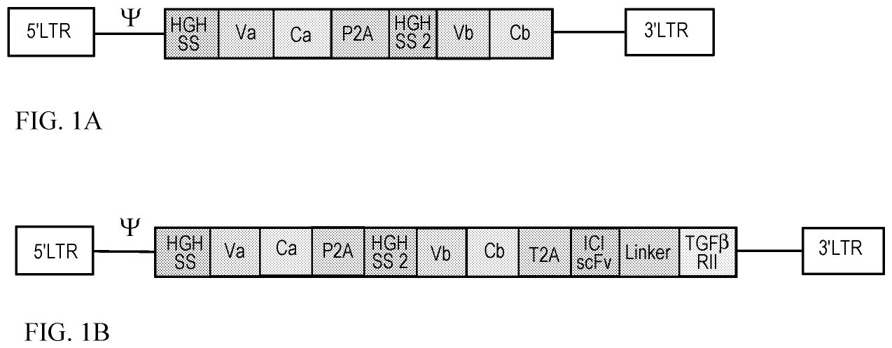

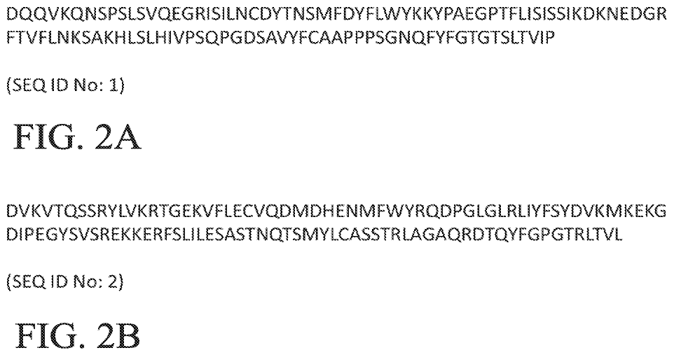

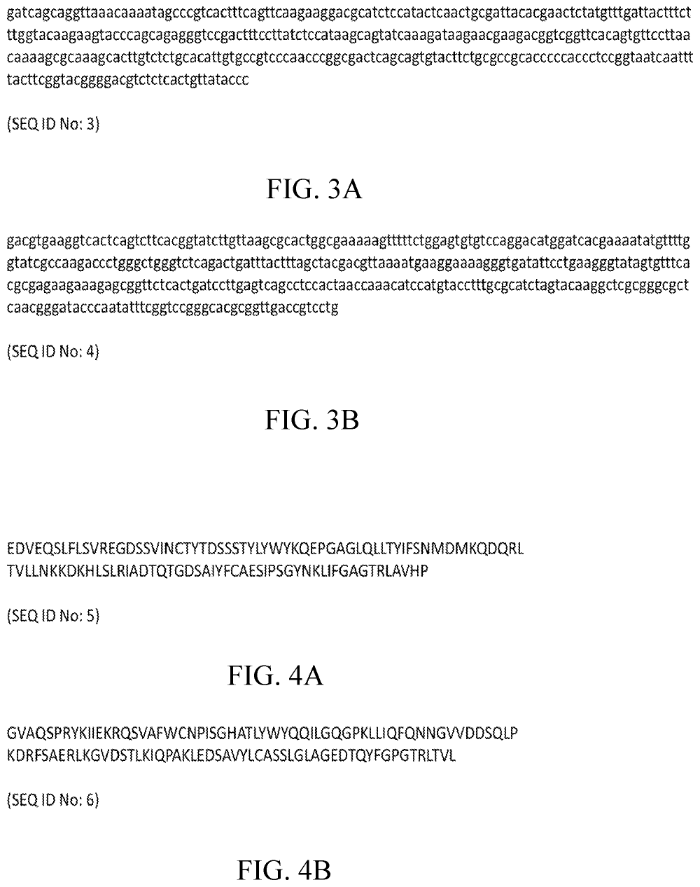

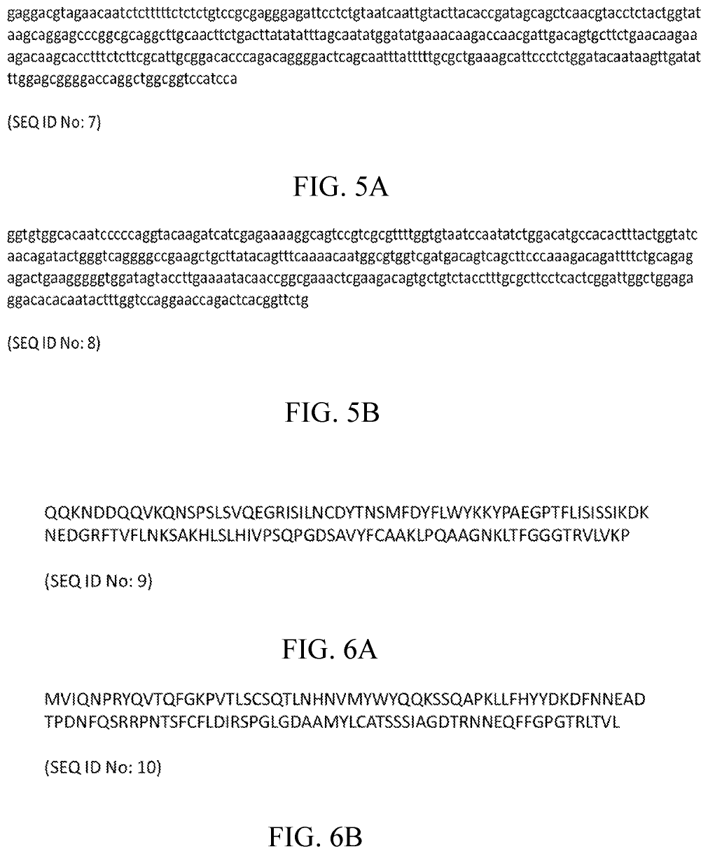

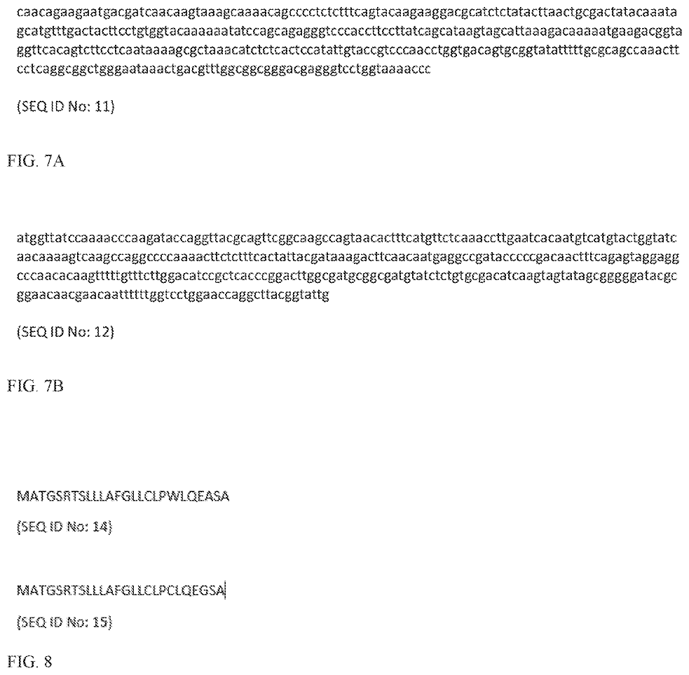

Referring to FIG. 1A, according to various preferred embodiments, the nucleic acid construct comprises two sequences, wherein the two sequences include: (a) the variable region of the alpha chain of an anti-LMP2 TCR fused to the constant region of a mouse TCR alpha chain identified as “aLMP-2_Va-Ca”, wherein aLMP-2_Va corresponds to the variable region of the alpha chain of an anti-LMP2 TCR and Ca corresponds to the constant region of a mouse TCR alpha chain; (b) the variable region of the beta chain of same anti-LMP2 TCR fused to the constant region of the mouse TCR beta chain identified as “aLMP-2_Vb-Cb”, wherein aLMP-2 Vb corresponds to the variable region of the beta chain of same human anti-LMP2 TCR and Cb corresponds to the constant region of the mouse TCR beta chain. In one embodiment, the nucleic acid construct further comprises a sequence encoding a signal peptide.

Referring to FIG. 1B, according to various embodiments, the nucleic acid construct comprises three sequences wherein the three sequences include: (a) the variable region of the alpha chain of a human TCR fused to the constant region of a mouse TCR alpha chain identified as “Va-Ca”, wherein Va corresponds to the variable region of the alpha chain of a human TCR and Ca corresponds to the constant region of a mouse TCR alpha chain; (b) the variable region of the beta chain of same human TCR fused to the constant region of the mouse TCR beta chain identified as “Vb-Cb”, wherein Vb corresponds to the variable region of the beta chain of same human TCR and Cb corresponds to the constant region of the mouse TCR beta chain; and, (c) the variable regions of the heavy and light chain of an immune checkpoint inhibitor (ICI), linked with a GS linker, fused to a ligand-binding sequence of the extracellular domain of TCRβRII via a flexible linker peptide at the C terminus of the variable region of the heavy chain. In preferred embodiments, the nucleic acid construct further comprises a sequence encoding a signal peptide. In some embodiments, the human TCR is an anti-LMP2 TCR. In some other embodiments, the human TCR is an anti-E-6 TCR. In some embodiments, the immune checkpoint inhibitor is an anti-PD-1 antibody. The variable regions of TCRs can be connected to signal peptide sequences.

The nucleic acid construct may further include other sequences which may assist and/or enable in the transfection, transduction, integration, replication, transcription, translation, expression and/or stabilization of the construct. In preferred embodiments, the nucleic acid construct comprises P2A and/or T2A sequences linking the supra mentioned sequences (a), (b) and/or (c).

The present disclosure also provides nucleic acids that encode TCR α and/or b chain as described herein. In some embodiments, the nucleic acid that encodes the a chain comprises the sequence set forth in any of SEQ ID NOs: 41, 43, or 45, or a nucleic acid sequence that has at least 80%, 85%, 90%, 91%, 92%, 93%, 94%, 95%, 96%, 97%, 98%, or 99% sequence identity thereto. In some embodiments, the nucleic acid that encodes the b chain comprises the sequence set forth in any of SEQ ID NOs: 42, 44, or 46, or a sequence that has at least 80%, 85%, 90%, 91%, 92%, 93%, 94%, 95%, 96%, 97%, 98%, or 99% sequence identity thereto. In some embodiments, the a chain comprises one or more Va CDR sequences as described herein. In some embodiments, the b chain comprises one or more Vb CDR sequences as described herein.

### Method for Preparation of Engineered Cells

The present invention provides a method or process for manufacturing and using the engineered cells for treatment of pathological diseases or conditions. The method comprises the steps of: (I) isolating the T cells from a patient's blood; (II) transducing the population T cells with a viral vector including the nucleic acid construct encoding a genetically engineered antigen receptor and an inhibitory protein; (III) expanding the transduced cells in vitro; and, (IV) infusing the expanded cells into the patient, where the engineered T cells will seek and destroy antigen positive tumor cells. In some embodiments, these engineered T cells can block PD-1/PD-L1 immunosuppression and strengthen the antitumor immune response.

The method further comprises: transfection of T cells with the viral vector containing the nucleic acid construct of the present invention, prior to step (II).

The transfection of T cells may be achieved by using any standard method such as calcium phosphate, electroporation, liposomal mediated transfer, microinjection, biolistic particle delivery system, or any other known methods by skilled artisan. In some embodiments, transfection of T cells is performed using the calcium phosphate method.

According to various embodiments described herein, the present invention provides an immunotherapy against tumors, particularly EBV and HPV associated cancers. The engineered T cells recognize a tumor associated HPV/EBV antigen and simultaneously secrete a single-chain antibody (scFv) fusion protein that blocks Programmed Cell Death Protein 1 (PD-1) and TGFβ. These engineered T cells demonstrate a stronger antitumor response and reduced T cell exhaustion.

It has been found experimentally that PD-1 checkpoint blockade is more effective with this invention because (1) anti-PD-1 drug delivery is localized to the tumor site and (2) the anti-PD-1 single-chain antibody binds more strongly than currently existing antibodies. Also, toxicity due to non-specific inflammation is reduced because anti-PD-1 drug delivery is localized to the tumor site. The present invention provides that combination of anti-LMP2 TCR and anti-PD-1 improves T cell activation and/or prevents T cell exhaustion compared to existing alternatives.

Also, the present invention provides a method to create a personalized anti-tumor immunotherapy. Anti-LMP2+/anti-PD-1 engineered T cells can be produced from a patient's blood. These engineered T cells are then reinfused into the patient as a cellular therapy product. This product could be applied to any patient who has an EBV LMP2 associated tumor, including, but are not limited to nasopharyngeal carcinoma, Hodgkin's lymphoma, Burkitt's lymphoma, stomach cancer and, others.

### Variants & Modifications

The binding molecule, e.g., TCR or antigen-binding fragment thereof, can be modified. In certain embodiments, the binding molecules, e.g., TCRs or antigen-binding fragments thereof, include one or more amino acid variations, e.g., substitutions, deletions, insertions, and/or mutations, compared to the sequence of a binding molecule, e.g., TCR, described herein. Exemplary variants include those designed to improve the binding affinity and/or other biological properties of the binding molecule. Amino acid sequence variants of a binding molecule may be prepared by introducing appropriate modifications into the nucleotide sequence encoding the binding molecule, or by peptide synthesis. Such modifications include, for example, deletions from, and/or insertions into and/or substitutions of residues within the amino acid sequences of the binding molecule. Any combination of deletion, insertion, and substitution can be made to arrive at the final construct, provided that the final construct possesses the desired characteristics, e.g., antigen-binding.

In some embodiments, one or more residues within a CDR of a parent binding molecule, e.g., TCR, is/are substituted. In some embodiments, the substitution is made to revert a sequence or position in the sequence to a germline sequence, such as a binding molecule sequence found in the germline (e.g., human germline), for example, to reduce the likelihood of immunogenicity, e.g., upon administration to a human subject.

The present disclosure also provides an antibody or antigen-binding fragment thereof that contains any one or more of the CDRs as described above with respect to TCRs. In some embodiments, the antibody or antigen-binding fragment contains variable heavy and light chain containing a CDR1, a CDR2 and/or a CDR3 contained in the alpha chain and a CDR1, a CDR2 and/or a CDR3 contained in the beta chain.

In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In some embodiments, the antibody light chain constant region is chosen from, e.g., kappa or lambda, particularly kappa. An antigen-binding fragment refers to a molecule other than an intact antibody that comprises a portion of an intact antibody that binds the antigen to which the intact antibody binds. Examples of antigen-binding fragment include but are not limited to Fv, Fab, Fab′, Fab′-SH, F(ab′)2; diabodies; linear antibodies; variable heavy chain (VH) regions, single-chain antibody molecules such as scFvs and single domain VH single antibodies; and multispecific antibodies formed from antibody fragments. In particular embodiments, the antibodies are single-chain antibody fragments comprising a variable heavy chain region and/or a variable light chain region, such as scFvs. Single-domain antibodies are antibody fragments comprising all or a portion of the heavy chain variable domain or all or a portion of the light chain variable domain of an antibody. In certain embodiments, a single-domain antibody is a human single-domain antibody.

In some embodiments, the antibody or antigen-binding portion thereof is expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against a peptide in the context of an MHC molecule also may be referred to as a TCR-like CAR. Thus, among the provided binding molecules, e.g., EBV binding molecules, are antigen receptors, such as those that include one of the provided antibodies, e.g., TCR-like antibodies. In some embodiments, the antigen receptors and other chimeric receptors specifically bind to a region or epitope of LMP2, e.g. TCR-like antibodies. Among the antigen receptors are functional non-TCR antigen receptors, such as chimeric antigen receptors (CARs). Also provided are cells expressing the CARs and uses thereof in adoptive cell therapy, such as treatment of diseases and disorders associated with HPV or EBV expression.

TCR-like CARs that contain a non-TCR molecule that exhibits T cell receptor specificity, such as for a T cell epitope or peptide epitope when displayed or presented in the context of an MHC molecule. In some embodiments, a TCR-like CAR can contain an antibody or antigen-binding portion thereof, e.g., TCR-like antibody, such as described herein. In some embodiments, the antibody or antibody-binding portion thereof is reactive against specific peptide epitope in the context of an MEW molecule, wherein the antibody or antibody fragment can differentiate the specific peptide in the context of the MHC molecule from the MHC molecule alone, the specific peptide alone, and, in some cases, an irrelevant peptide in the context of an MEW molecule. In some embodiments, an antibody or antigen-binding portion thereof can exhibit a higher binding affinity than a T cell receptor.

Exemplary antigen receptors, including CARs, and methods for engineering and introducing such receptors into cells, include those described, for example, in U.S. patent application publication numbers US2002/131960, US2013/287748, US2013/0149337, U.S. Pat. Nos. 6,451,995, 7,446,190, 8,252,592; each of which is incorporated herein by reference in its entirety.

In some embodiments, the CARs generally include an extracellular antigen (or ligand) binding domain, including as an antibody or antigen-binding fragment thereof specific for a peptide in the context of an MEW molecule, linked to one or more intracellular signaling components, in some aspects via linkers and/or transmembrane domain(s). In some embodiments, such molecules can typically mimic or approximate a signal through a natural antigen receptor, such as a TCR, and, optionally, a signal through such a receptor in combination with a co stimulatory receptor.

In some embodiments, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAh). In some embodiments, the CAR contains a TCR-like antibody, such as an antibody or an antigen-binding fragment (e.g., scFv) that specifically recognizes a peptide epitope presented on the cell surface in the context of an MEW molecule.

### Bifunctional Trap Fusion Protein

The present disclosure also provides bifunctional trap fusion proteins. Monoclonal antibodies targeting immune checkpoints (e.g., PD-1 or PD-L1) are a major class of these agents. The PD-1 receptor is expressed on activated T and natural killer (NK) cells. After interaction with its ligands PD-L1 and PD-L2, which are typically expressed on antigen presenting cells, PD-1 regulates immune responses by inhibiting T and NK cell maturation, proliferation, and effector function.

In addition to expression of immune checkpoints, the tumor microenvironment contains other immunosuppressive molecules. Of particular interest is the cytokine TGF-β (TGFB), which has multiple functions in cancer. TGF-β prevents proliferation and promotes differentiation and apoptosis of tumor cells early in tumor development. However, during tumor progression, tumor TGF-β insensitivity arises due to the loss of TGF-β receptor expression or mutation to downstream signaling elements. TGF-β then promotes tumor progression through its effects on angiogenesis, induction of epithelial-to-mesenchymal transition (EMT), and immune suppression. High TGF-β serum level and loss of TGF-β receptor (TGFβR) expression on tumors correlates with poor prognosis. TGFβ-targeted therapies have demonstrated limited clinical activity.

In some aspects, the present disclosure provides bifunctional trap proteins that can target both immune checkpoints and TGF-β negative regulatory pathways. In some embodiments, the bifunctional trap protein targets both the PD-1 and TGF-β. In some embodiments, the bifunctional trap protein targets both the PD-L1 and TGF-β. In some embodiments, the bifunctional fusion protein designed to block PD-L1 and sequester TGF-β. M7824 (MSB0011395C) comprises the extracellular domain of human TGF-β receptor II (TGFβRII) linked to the C-terminus of the human anti-PD-L1 scFv, based on the human IgG1 monoclonal antibody (mAb) avelumab. In some embodiments, the bifunctional fusion protein comprises the extracellular domain of human TGF-β receptor II (TGFβRII) linked to the C-terminus of the human anti-PD-1 scFv.

These bifunctional trap fusion proteins are described e.g., Knudson, et al. “M7824, a novel bifunctional anti-PD-L1/TGFβ Trap fusion protein, promotes anti-tumor efficacy as monotherapy and in combination with vaccine.” Oncoimmunology 7.5 (2018): e1426519, which is incorporated herein by reference in its entirety.

The present disclosure provides methods of treating various disorders as described herein (e.g., cancer) by using TCR or antigen-binding molecules as described herein in combination with one or more bifunctional trap fusion proteins. In some embodiments, the subject is treated by cells that express TCR or antigen-binding molecules as described herein and one or more bifunctional trap fusion proteins.

### Compositions, Formulations and Methods of Administration

The present disclosure provides compositions (including pharmaceutical and therapeutic compositions) containing the engineered T cells and populations thereof, produced by the disclosed methods. Also provided are methods, e.g., therapeutic methods for administrating the engineered T cells and compositions thereof to subjects, e.g., patients.

A. Compositions and Formulations

Compositions including the engineered T cells for administration, including pharmaceutical compositions and formulations, such as unit dose form compositions including the number of cells for administration in a given dose or fraction thereof are provided. The pharmaceutical compositions and formulations may include one or more optional pharmaceutically acceptable carrier or excipient. In some embodiments, the composition includes at least one additional therapeutic agent.

In some embodiments, the choice of carrier is determined in part by the particular cell (e.g., T cell or NK cell) and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some embodiments, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Suitable buffering agents used in the invention include, for example, citric acid, sodium citrate, phosphoric acid, potassium phosphate, and various other acids and salts. In some embodiments, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for a particular indication, disease, or condition being treated with the engineered T cells, preferably those with activities complementary to the cells, where the respective activities do not adversely affect one another. Such active ingredients are suitably present in combination in amounts that are effective for the purpose intended. Thus, in some embodiments, the pharmaceutical composition may further include other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/orvincristine.

The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

The cells and compositions may be administered using standard administration techniques, formulations, and/or devices. Administration of the cells can be autologous or heterologous. For example, immunoresponsive T cells or progenitors can be obtained from one subject, and administered to the same subject or a different, compatible subject after genetically modifying them in accordance with various embodiments described herein. Peripheral blood derived immunoresponsive T cells or their progeny (e.g., in vivo, ex vivo or in vitro derived) can be administered via localized injection, including catheter administration, systemic injection, localized injection, intravenous injection, or parenteral administration. Usually, when administering a therapeutic composition (e.g., a pharmaceutical composition containing a genetically modified immunoresponsive cell), it is generally formulated in a unit dosage injectable form (solution, suspension, emulsion).

Formulations disclosed herein include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

The compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

B. Methods of Administration and Uses of Engineered T Cells in Adoptive Cell Therapy

Provided are methods of administering the cells, populations, and compositions, and uses of such cells, populations, and compositions to treat or prevent diseases, conditions, and disorders, including cancers. In some embodiments, the methods described herein can reduce the risk of the developing diseases, conditions, and disorders as described herein.

In some embodiments, the cells, populations, and compositions, described herein are administered to a subject or patient having a particular disease or condition to be treated, e.g., via adoptive cell therapy, such as adoptive T cell therapy. In some embodiments, cells and compositions prepared by the provided methods, such as engineered compositions and end-of-production compositions following incubation and/or other processing steps, are administered to a subject, such as a subject having or at risk for the disease or condition. In some aspects, the methods thereby treat, e.g., ameliorate one or more symptom of, the disease or condition, such as by lessening tumor burden in cancer expressing an antigen recognized by the engineered T cells.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(14577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive T cell therapy, is carried out by autologous transfer, in which the T cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the T cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at high risk of relapse, and thus the cells are administered prophylactically, e.g., to reduce the likelihood of or prevent relapse. In some embodiments, the subject has not received prior treatment with another therapeutic agent.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8+ and CD4+ T cells, are administered at or within a tolerated difference of a desired dose of total cells, such as a desired dose of T cells. In some embodiments, the desired dose is a desired number of cells or a desired number of cells per unit of body weight of the subject to whom the cells are administered, e.g., cells/kg. In some embodiments, the desired dose is at or above a minimum number of cells or minimum number of cells per unit of body weight. In some embodiments, among the total cells, administered at the desired dose, the individual populations or sub-types are present at or near a desired output ratio (such as CD4+ to CD8+ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some embodiments, the desired dose is a desired number of cells of the sub-type or population, or a desired number of such cells per unit of body weight of the subject to whom the cells are administered, e.g., cells/kg. In some embodiments, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4+ to CD8+ cells, and/or is based on a desired fixed or minimum dose of CD4+ and/or CD8+ cells. In certain embodiments, the cells or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 10⁴ and at or about 10⁹ cells/kilograms (kg) body weight, such as between 10⁵ and 10⁶ cells/kg body weight, for example, at least or at least about or at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at least or at least about or at or about 1×10⁵ T cells/kg, 1.5×10⁵ T cells/kg, 2×10⁵ T cells/kg, or 1×10⁶ T cells/kg body weight.

In some embodiments, the cells are administered at or within a certain range of error of between at or about 10⁴ and at or about 10⁹ CD4+ and/or CD8+ cells/kilograms (kg) body weight, such as between 10⁵ and 10⁶ CD4+ and/or CD8+ cells/kg body weight, for example, at least or at least about or at or about 1×10⁵ CD4+ and/or CD8+ cells/kg, 1.5×10⁵ CD4+ and/or CD8+ cells/kg, 2×10⁵ CD4+ and/or CD8+ cells/kg, or 1×10⁶ CD4+ and/or CD8+ cells/kg body weight.

In some embodiments, the cells are administered at or within a certain range of error of, greater than, and/or at least about 1×10⁶, about 2.5×10⁶, about 5×10⁶, about 7.5×10⁶, or about 9×10⁶ CD4+ cells, and/or at least about 1×10⁶, about 2.5×10⁶, about 5×10⁶, about 7.5×10⁶, or about 9×10⁶ CD8+ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4+ cells, and/or between about 10⁸ and 10¹² or between about 10¹⁰ and 10¹¹ CD8+ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4+ to CD8+ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

The cells described herein can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

Following administration of the cells, the biological activity of the engineered cell populations in some embodiments is measured, e.g., by any of a number of known methods. Parameters to assess include specific binding of engineered T cells to the antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

In certain embodiments, the engineered cells are further modified in any number of ways, such that their therapeutic or prophylactic efficacy is increased. For example, the engineered CAR or TCR expressed by the population can be conjugated either directly or indirectly through a linker to a targeting moiety. The practice of conjugating compounds, e.g., the CAR or TCR, to targeting moieties is known in the art. See, for instance, Wadwa et al., J. Drug Targeting 3: 111 (1995), and U.S. Pat. No. 5,087,616.

C. Dosing Schedule or Regimen

In some embodiments, repeated dosage methods are provided in which a first dose of cells is given followed by one or more second consecutive doses. The timing and size of the multiple doses of cells generally are designed to increase the efficacy and/or activity and/or function of TCR-expressing engineered T cells, when administered to a subject in adoptive therapy methods. In some embodiments, the repeated dosings reduce the downregulation or inhibiting activity that can occur when inhibitory immune molecules, such as PD-1 and/or PD-L1 are upregulated on TCR-expressing engineered T cells. The methods involve administering a first dose, generally followed by one or more consecutive doses, with particular time frames between the different doses.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time, which is no more than 3 days. Thus, in some contexts, the first or consecutive dose is a single or continuous administration of the specified number of cells, given or initiated at a single point in time. In some contexts, however, the first or consecutive dose is administered in multiple injections or infusions over a period of no more than three days, such as once a day for three days or for two days or by multiple infusions over a single day period.

Thus, in some aspects, the cells of the first dose are administered in a single pharmaceutical composition. In some embodiments, the cells of the consecutive dose are administered in a single pharmaceutical composition.

In some embodiments, the cells of the first dose are administered in a plurality of compositions, collectively containing the cells of the first dose. In some embodiments, the cells of the consecutive dose are administered in a plurality of compositions, collectively containing the cells of the consecutive dose. In some aspects, additional consecutive doses may be administered in a plurality of compositions over a period of no more than 3 days.

The term “split dose” refers to a dose that is split so that it is administered over more than one day. This type of dosing is encompassed by the present methods and is considered to be a single dose.

Thus, the first dose and/or consecutive dose(s) in some aspects may be administered as a split dose. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the first dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

With reference to a prior dose, such as a first dose, the term “consecutive dose” refers to a dose that is administered to the same subject after the prior, e.g., first, dose without any intervening doses having been administered to the subject in the interim. Nonetheless, the term does not encompass the second, third, and/or so forth, injection or infusion in a series of infusions or injections comprised within a single split dose. Thus, unless otherwise specified, a second infusion within a one, two or three-day period is not considered to be a “consecutive” dose as used herein. Likewise, a second, third, and so-forth in the series of multiple doses within a split dose also is not considered to be an “intervening” dose in the context of the meaning of “consecutive” dose. Thus, unless otherwise specified, a dose administered a certain period of time, greater than three days, after the initiation of a first or prior dose, is considered to be a “consecutive” dose even if the subject received a second or subsequent injection or infusion of the cells following the initiation of the first dose, so long as the second or subsequent injection or infusion occurred within the three-day period following the initiation of the first or prior dose.

Thus, unless otherwise specified, multiple administrations of the same cells over a period of up to 3 days is considered to be a single dose, and administration of cells within 3 days of an initial administration is not considered a consecutive dose and is not considered to be an intervening dose for purposes of determining whether a second dose is “consecutive” to the first.

In some embodiments, multiple consecutive doses are given, in some aspects using the same timing guidelines as those with respect to the timing between the first dose and first consecutive dose, e.g., by administering a first and multiple consecutive doses, with each consecutive dose given within a period of time in which an inhibitory immune molecule, such as PD-1 and/or PD-L1, has been upregulated in cells in the subject from an administered first dose. It is within the level of a skilled artisan to empirically determine when to provide a consecutive dose, such as by assessing levels of PD-1 and/or PD-L1 in antigen-expressing, such as TCR-expressing cells, from peripheral blood or other bodily fluid.

In some embodiments, the timing between the first dose and first consecutive dose, or a first and multiple consecutive doses, is such that each consecutive dose is given within a period of time is greater than about 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days or more. In some embodiments, the consecutive dose is given within a time period that is less than about 28 days after the administration of the first or immediately prior dose. The additional multiple additional consecutive dose or doses also are referred to as subsequent dose or subsequent consecutive dose.

The size of the first and/or one or more consecutive doses of cells are generally designed to provide improved efficacy and/or reduced risk of toxicity. In some aspects, a dosage amount or size of a first dose or any consecutive dose is any dosage or amount as described above. In some embodiments, the number of cells in the first dose or in any consecutive dose is between about 0.5×10⁶ cells/kg body weight of the subject and 5×10⁶ cells/kg, between about 0.75×10⁶ cells/kg and 3×10⁶ cells/kg or between about 1×10⁶ cells/kg and 2×10⁶ cells/kg, each inclusive.

As used herein, “first dose” is used to describe the timing of a given dose being prior to the administration of a consecutive or subsequent dose. The term does not necessarily imply that the subject has never before received a dose of cell therapy or even that the subject has not before received a dose of the same cells or cells expressing the same recombinant receptor or targeting the same antigen.

In some embodiments, the receptor, e.g., the TCR, expressed by the cells in the consecutive dose contains at least one immunoreactive epitope as the receptor, e.g., the TCR, expressed by the cells of the first dose. In some embodiments, the receptor, e.g., the TCR, expressed by the cells administered in the consecutive dose is identical to the receptor, e.g., the TCR, expressed by the first dose or is substantially identical to the receptor, e.g., the TCR, expressed by the cells of administered in the first dose.

The receptors, such as TCRs, expressed by the cells administered to the subject in the various doses generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells in the first dose express a TCRs that specifically binds to an antigen expressed.

### EXAMPLES

The following examples are not intended to limit the scope of the claims to the invention, but is rather intended to be exemplary of certain embodiments. Any variations in the exemplified methods which occur to the skilled artisan are intended to fall within the scope of the present invention.

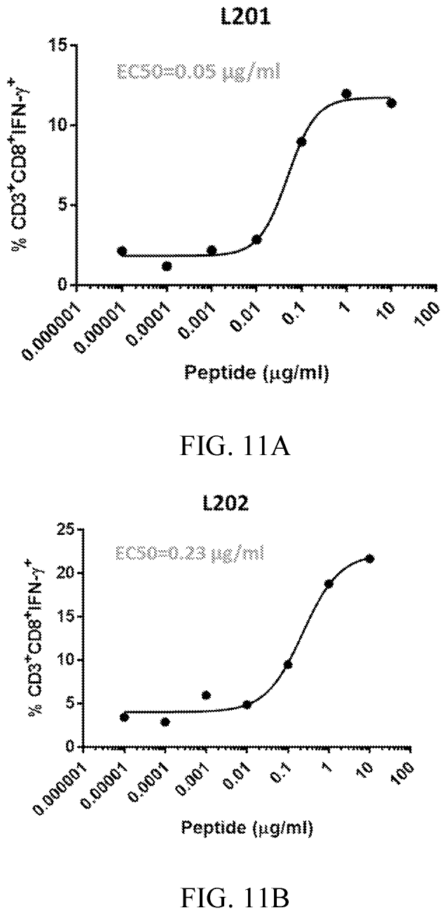

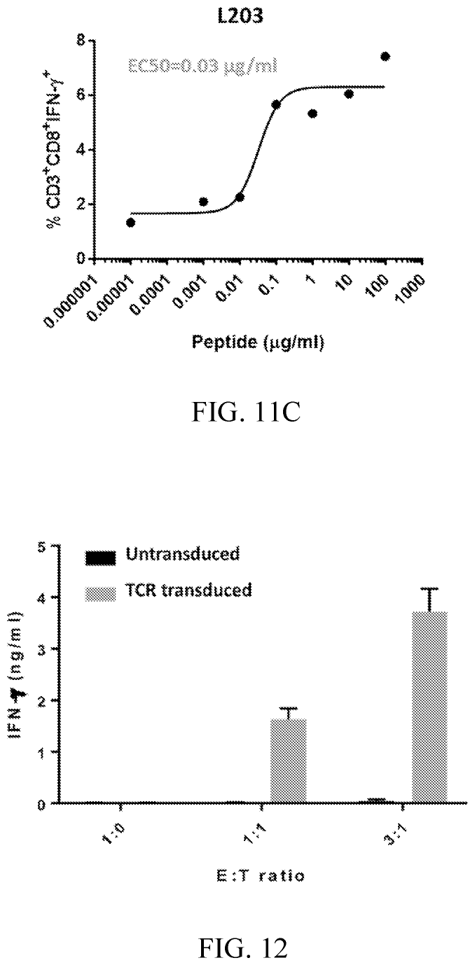

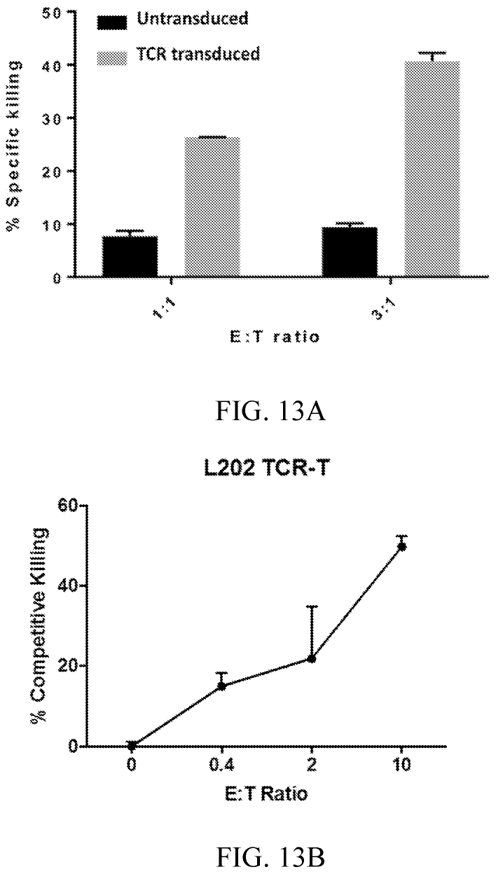

Cancers commonly associated with infection by viruses, including Epstein Barr virus (EBV) and human papillomavirus (HPV), are excellent targets for adoptive immunotherapy. Here, we identified a novel T cell receptor (TCR) sequence capable of activating in response to EBV's latent membrane protein 2 (LMP2) antigen (TCR-L201; FIGS. 10 and 11). Consistent with these interferon gamma (IFNγ) activation results, T cells expressing TCR-L201 can specifically kill cancer cells engineered to express an LMP2 peptide linked to HLA-A2 (FIG. 13A). Because HLA-A2 is among the most common human serotypes, the L201 TCR has utility for engineered TCR-T cell therapy against EBV-associated NPC as well as lymphomas including Hodgkin's and Burkitt's.

### Construct Design.

For LMP2 TCR-T cells, an MP71 retroviral vector construct containing 2 coding regions was generated using standard molecular biology techniques: (1) the variable region of the alpha chain of a human anti-LMP2 TCR fused to the constant region of the mouse TCR alpha chain; (2) the variable region of the beta chain of same human anti-LMP2 TCR fused to the constant region of the mouse TCR beta chain. (FIG. 1A)

For TCR-ICI-TGFβTRAP TCR-T cells, an MP71 retroviral vector construct containing 3 coding regions was generated using standard molecular biology techniques: (1) the variable region of the alpha chain of a human specific TCR fused to the constant region of the mouse TCR alpha chain; (2) the variable region of the beta chain of same human TCR fused to the constant region of the mouse TCR beta chain; (3) the variable regions of the heavy and light chain of an immune checkpoint inhibitor (ICI) linked with a GS linker, fused to a ligand-binding sequence of the extracellular domain of TCRβRII via a flexible linker peptide at the C terminus of the variable heavy chain. Anti-gp120-TCRβRII antibody is used as a non-specific scFv-TCRβRII control. (FIG. 1B)

### Cell Lines and Media

HEK-293T, Ca Ski, and K562 cells were purchased from ATCC. Peripheral blood mononuclear cells (PBMCs) from anonymous donors were purchased from Hemacare. K562-A2 cells were produced by lentiviral transduction of K562 cells with a vector overexpressing human HLA-A2 single chain. Ca Ski E6/E7 cells were produced by retroviral transduction of Ca Ski cells with a vector overexpressing human E6 and E7. A375-pHLA (LLW) and A375-pHLA (CLG) cells were produced by retroviral transduction of vector overexpression LLW epitope-linker-HLA-A2 or CLG epitope-linker-HLA-A2. Cells were cultured in DMEM+10% FBS, RPMI+10% FBS, or X-Vivo+5% human serum A/B.

### Retroviral Vector Production

Retroviral vectors were prepared by transient transfection of HEK-293T cells using a standard calcium phosphate precipitation protocol. Viral supernatants were harvested at 48h and used to transduce T cells. T cell transduction and expansion. Before retroviral transduction, PBMCs were activated for 2 days by culturing with T cell activator beads and human IL-2. For transduction, freshly harvested retroviral supernatant was spin-loaded onto non-tissue culture-treated 24-well plates coated with 15 mg RetroNectin per/well (Clontech Laboratories) by centrifuging 2 hr at 2,000 g at 32° C. Activated PBMCs were loaded onto the plates and spun at 600 g at 32° C. for 30 min. T cells were incubated at 37° C. and 5% CO₂. Culture medium was replenished every 2 days.

### TCR Staining

All antibodies were purchased from Biolegend. Expression of the recombinant TCR was detected 72h after transfection by antibody staining to mouse TCR beta chain followed by flow cytometry. CD3, CD4, and CD8 staining was performed simultaneously. A viable CD3+ lymphocyte gating strategy was used. NT=non-transduced control.

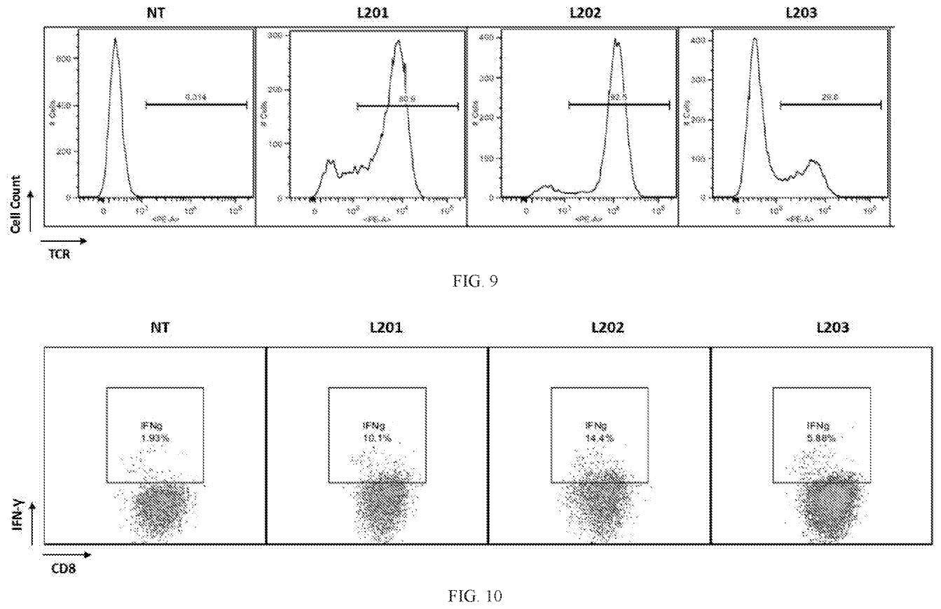

Primary human T cells were transduced with the constructs of L201, L202 and L203 TCR. Results: (FIG. 9) The anti-LMP2 TCR is expressed strongly in human T cells.

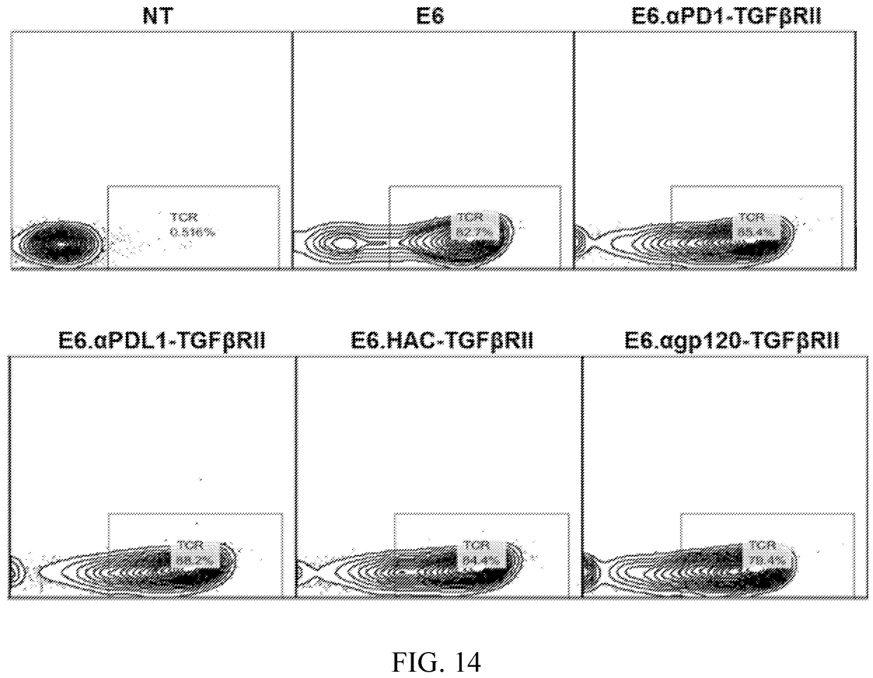

Primary human T cells were transduced with the constructs of E6, E6-αPD1-TGFβRII, E6-αPDL1-TGFβRII, E6-HAC-TGFβRII or E6-αgp120-TGFβRII TCR. Results: (FIG. 14) The anti-E6 TCR is expressed strongly in T cells containing the original anti-E6 TCR, the E6-αPD1-TGFβRII, E6-αPDL1-TGFβRII, E6-HAC-TGFβRII and the E6-αgp120-TGFβRII TCR construct.

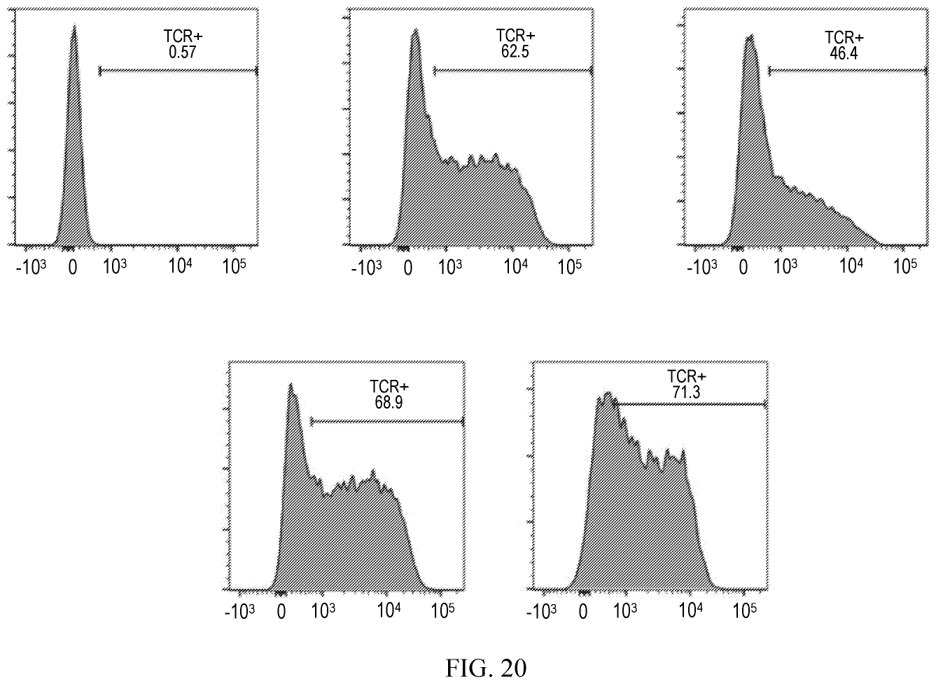

Primary human T cells were transduced with the constructs of LMP2-αPD1-TGFβRII, LMP2-αPDL1-TGFβRII, LMP2-HAC-TGFβRII or LMP2-αgp120-TGFβRII TCR. Results: (FIG. 20) The anti-LMP2 TCR is expressed strongly in T cells containing the original anti-LMP2 TCR, the LMP2-αPD1-TGFβRII, LMP2-αPDL1-TGFβRII, LMP2-HAC-TGFβRII and the LMP2-αgp120-TGFβRII TCR construct.

### In Vitro TCR-T IFNβ Production.

TCR-T cells were cocultured with different types of target cells at various effector-to-target ratios, as indicated. Intracellular or secreted IFN-γ expression was measured by flow cytometry or with a human IFN-γ ELISA kit according to the manufacturer's instructions, respectively.

TCR-T cells with anti-LMP2 TCRs were cocultured for overnight with EBV peptide-pulsed APCs at 1:1 effector-to-target ratios. Results: (FIGS. 10 and 11A-11C) TCR-T cells containing the anti-LMP2 TCR could be specifically activated by target cells, as measured by intracellular IFN-γ expression. All three anti-LMP2 TCRs showed sub-micromolarEC50.

L201 TCR-T cells were cocultured for 48 hrs with EBV peptide-pulsed APCs at 1:0, 1:1, and 3:1 effector-to-target ratios. Results: (FIG. 12) TCR-T cells could be activated by target cells. Higher E:T ratio leads the TCR-T cells to produce more IFN-γ.

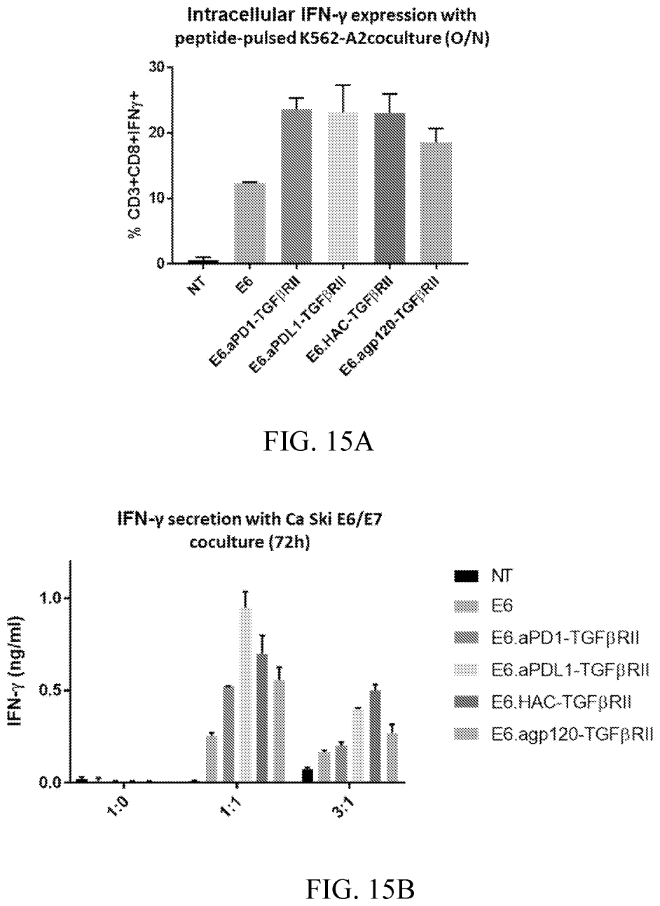

The effects of secreted ICI-TGFβRII traps on IFNγ production of TCR-T cells upon antigen-specific stimulation. (a) TCR-T cells were cocultured for overnight with peptide-pulsed K562-A2 cells at 1:1 effector-to-target ratio. The cells were then collected and intracellular IFN-γ expression was measured by flow cytometry. (FIG. 15A) (b) TCR-T cells were cocultured for 72 hrs with Ca Ski E6/E7 cells at 1:0, 1:2, 1:1, and 3:1 effector-to-target ratios (FIG. 15B). The supernatant was then collected and the IFN-γ production was measured using a human IFN-γ ELISA kit according to the manufacturer's instructions. Results: (FIG. 15B) TCR-T cells containing the E6 TCR could be activated by target cells, as measured by IFN-γ expression. Stimulated either by peptide-pulsed APCs or E6+ target cells (Ca Ski E6/E7), the E6-αPD1-TGFβRII, E6-αPDL1-TGFβRII, E6-HAC-TGFβRII or E6-αgp120-TGFβRII TCR-T cells have much higher IFN-γ expression than E6 alone. Compared to control E6-αgp120-TGFβRII TCR-T cells, E6-αPD1-TGFβRII, E6-αPDL1-TGFβRII, E6-HAC-TGFβRII TCR-T cells produce higher levels of IFN-γ upon antigen-specific stimulation.

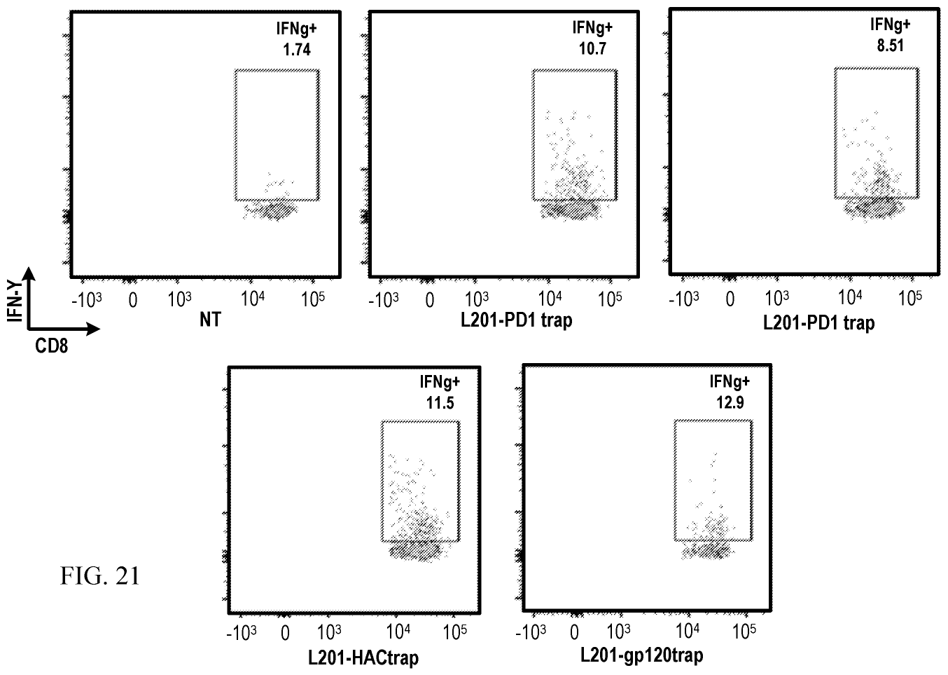

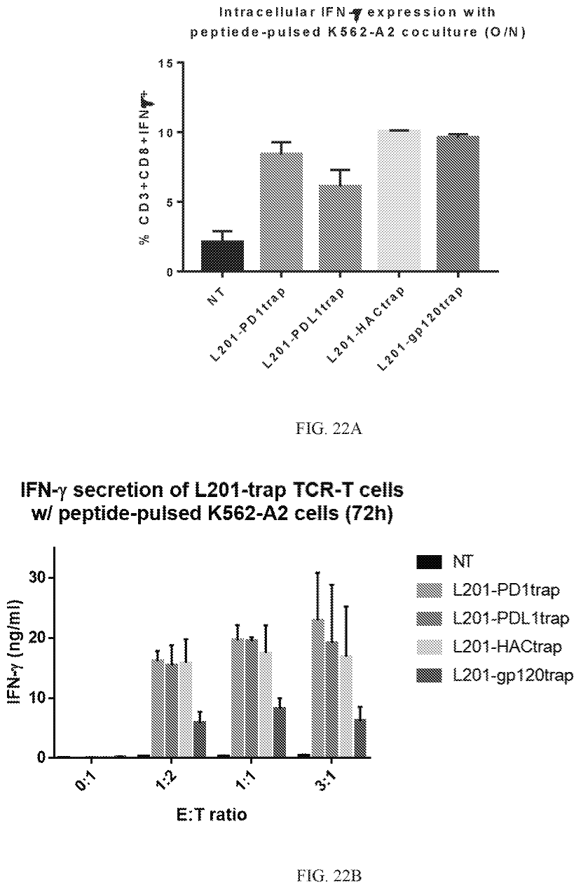

LMP2-αPD1-TGFβRII, LMP2-αPDL1-TGFβRII, LMP2-HAC-TGFβRII or LMP2-αgp120-TGFβRII TCR-T cells were cocultured for overnight with LMP2-LLW peptide-pulsed APCs at 1:1 effector-to-target ratios. Results: (FIG. 21) TCR-T cells containing the LMP2 TCR could be activated by target cells, as measured by IFN-γ expression. Stimulated by peptide-pulsed APCs, the LMP2 alone, LMP2-αPD1-TGFβRII, LMP2-αPDL1-TGFβRII, LMP2-HAC-TGFβRII and LMP2-αgp120-TGFβRII TCR-T cells have high IFN-γ expression.

### Specific Cell Lysis (Cytotoxicity)

For LMP2 TCR-T cell killing assays, EBV peptide-pulsed APCs (K562-A2) were pre-stained with CFSE and then cocultured for overnight with untransduced or TCR transduced T cells at 1:1, and 3:1 effector-to-target ratios. The cytotoxicity of T cells against target cells was measured by Annexin V/7-AAD staining. For A375 cell killing, target (A375-pHLA(LLW)) and non-target (A375-pHLA(CLG)) cells were labeled with CFSE and Celltrace Violet, respectively, and mixed at a 1:1 ratio. Mixed cells were then co-cultured overnight with L202 TCR-T cells at various effector-to target cell ratios. The cytotoxicity of T cells against target cells was measured by the ratio of target to non-target cells.

Cytotoxicity of L201 TCR-T cells or L202 TCR-T cells against target cells. (a) EBV peptide APCs were pre-stained with CFSE and then cocultured for overnight with L201 TCR-T cells at 1:1 and 3:1 effector-to-target ratios. The cytotoxicity of T cells against target cells was measured by Annexin V/7-AAD staining. Results: (FIG. 13A) L201 anti-LMP2 TCR-T cells killed the target cells in a specific manner. With higher E:T ratio, the TCR-T cells have higher killing capacity.

(b) Target (A375-pHLA(LLW)) and non-target (A375-pHLA(CLG)) cells were labeled with CFSE and Celltrace Violet, respectively, and mixed at a 1:1 ratio. Mixed cells were then co-cultured overnight with L202 TCR-T cells at the indicated effector-to target cell ratios. Results: (FIG. 13B) L202 anti-LMP2 TCR-T cells killed the target cells in a specific manner. With higher E:T ratio, the TCR-T cells have higher killing capacity.

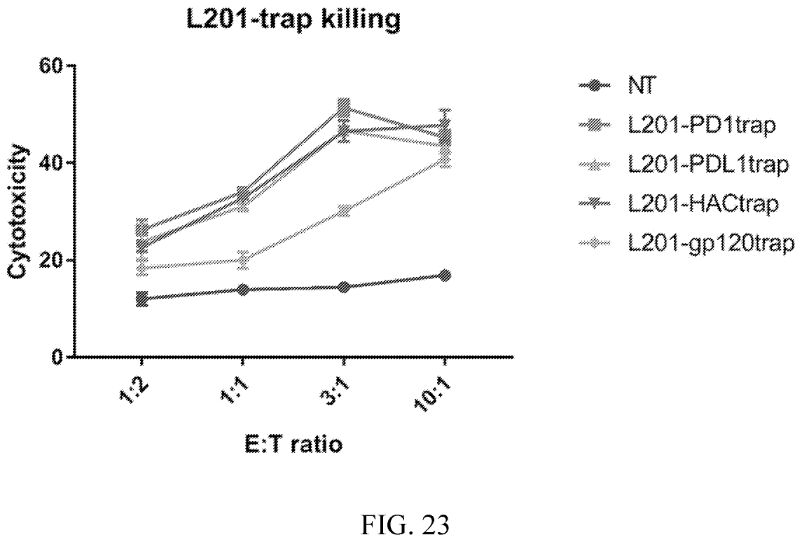

The specific killing of various anti-LMP2 TCR-T cells towards target cells. LMP2-LLW peptide pulsed APCs were pre-stained with CFSE and then cocultured overnight with TCR-T cells at multiple effector-to-target ratios. The cytotoxicity of T cells against LMP2-LLW peptide pulsed APCs was measured by Annexin V/7-AAD staining. Results: (FIG. 23) All LMP2 TCR-T cells killed LMP2+ target cells (Ca Ski) in a specific manner. Control LMP2.αgp120-TGFβRII TCR-T cells killed target cells more weakly than the other LMP2 TCR-T cells. Thus, LMP2-αPD1-TGFβRII, LMP2-αPDL1-TGFβRII, LMP2-HAC-TGFβRII TCR-T cells have higher killing capacity than the LMP2-αgp120-TGFβRII TCR-T cells.

For E6-ICI-TGFbTRAP T cell killing assays, Ca Ski tumor cells were pre-stained with CFSE and then cocultured for overnight with E6, E6.αPD1-TGFβRII, E6.αPDL1-TGFβRII, E6.HAC-TGFβRII or E6.αgp120-TGFβRII TCR-T cells at 1:1 effector-to-target ratio. The cytotoxicity of T cells against Ca Ski7 cells was measured by Annexin V/7-AAD staining.

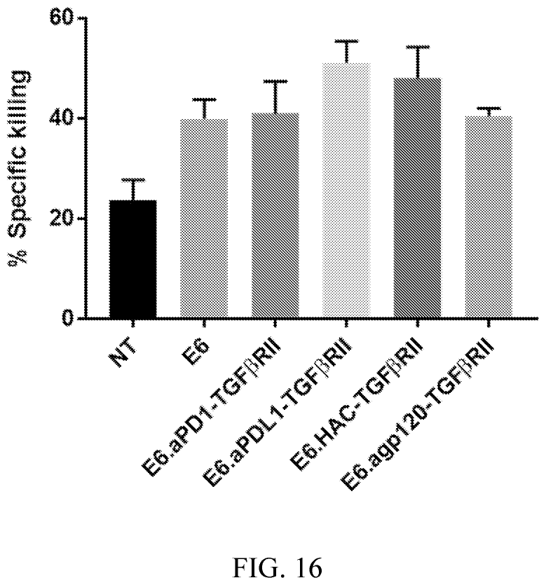

Results: (FIG. 16) All E6 TCR-T cells killed E6+ target cells (Ca Ski) in a specific manner. E6.αgp120-TGFβRII TCR-T killed target cells as efficiently as E6 alone, and E6.αPDL1-TGFβRII, E6.HAC-TGFβRII TCR-T cells have higher killing capacity than the E6.αgp120-TGFβRII TCR-T cells.

Binding activity of secreted scFv-TGFβRII to TGFβ. Recombinant human TGFβ1 was added to plates coated with scFv-TGFβRII, which was detected by biotinylated anti-TGFβ1 and HRP-Avidin.

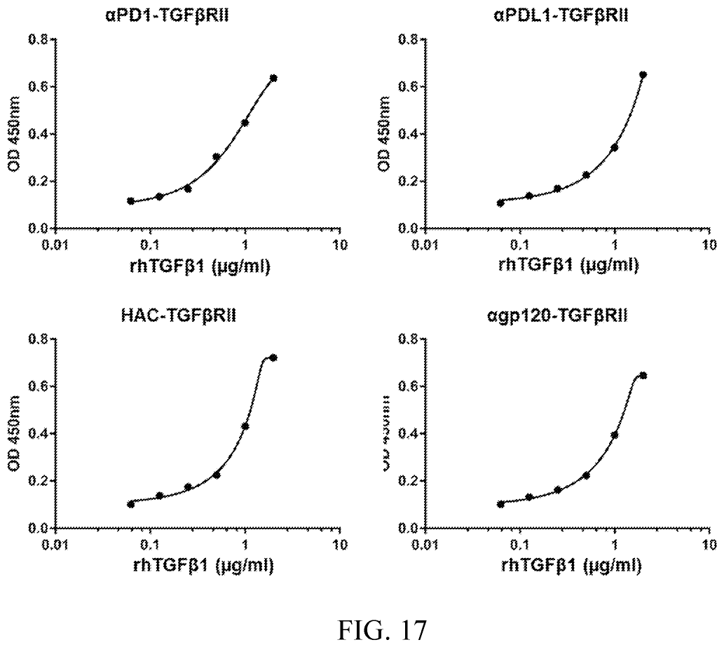

Results: (FIG. 17) The secreted scFv-TGFβRII produced by E6.αPD1-TGFβRII, E6.αPDL1-TGFβRII, E6.HAC-TGFβRII or E6.αgp120-TGFβRII TCR transfected 293T cells binds to recombinant human TGFβ2 at similar affinity.

TGFβ expression. The secreted TGFβ in E6+ target cells (Ca Ski) was measured using a human TGFβ ELISA kit according to the manufacturer's instructions.

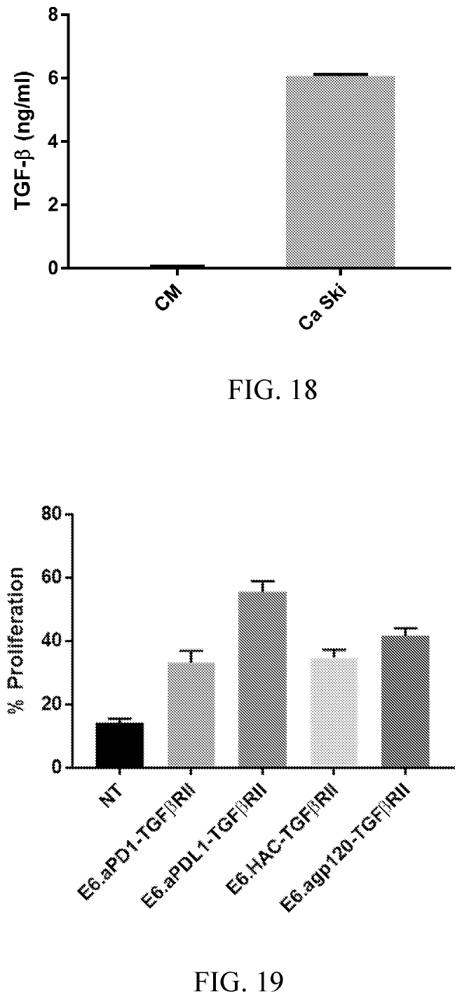

Results: (FIG. 18) The E6+ target cells (Ca Ski) can produce and secrete TGFβ into the supernatant.

In vitro TCR-T cell proliferation. E6, E6.αPD1-TGFβRII, E6.αPDL1-TGFβRII, E6.HAC-TGFβRII or E6.αgp120-TGFβRII TCR-T cells were pre-stained with CFSE. The stained T cells were then cocultured for 72 hours with Ca Ski cells and the intensity of CFSE was measured by flow cytometry. Nontransduced (NT) T cells were used as a control.

Results: (FIG. 19) Exposure to E6+ target cells stimulated all E6 TCR-T cells to proliferate, another measure of activation. E6.αPDL1-TGFβRII TCR-T cells proliferated faster than the other TCR-T cells tested.

### In Vivo Antitumor Efficacy of L202 TCR-T Cells.

Method: 6-8-week-old female NSG mice were inoculated with 5.0×10⁶ A375-pep-HLA-A2 melanoma cells subcutaneously in the right flank. 9 days later, on study Day 0, animals were sorted into groups based on tumor volume with each group bearing an average tumor volume of 35 mm³. On study Day 0 animals were intravenously injected with 10×10⁶ TCR+L202 cells or untransduced cells. These injections were repeated 7 days later, on study Day 6.

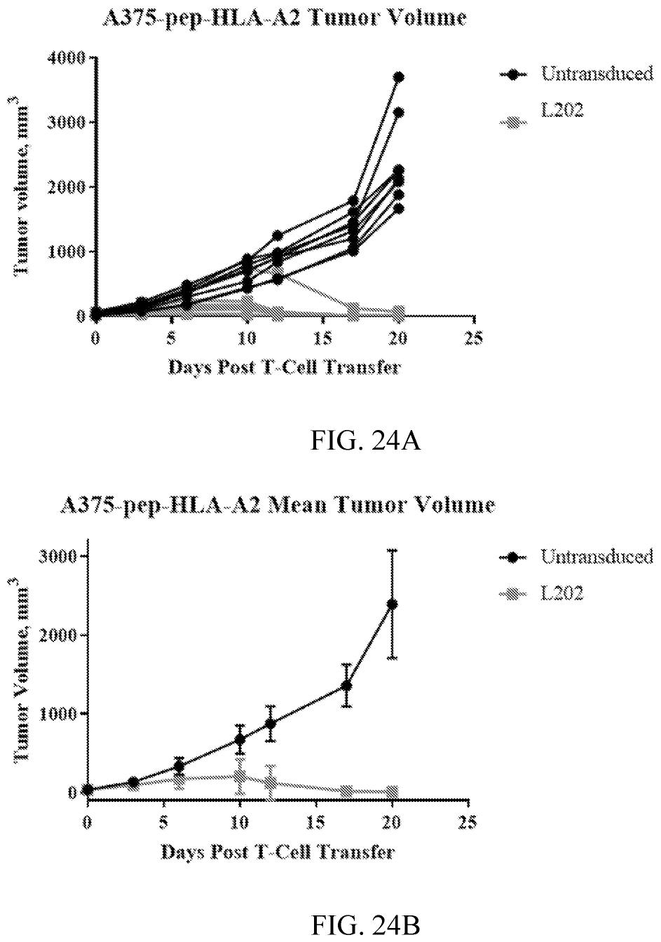

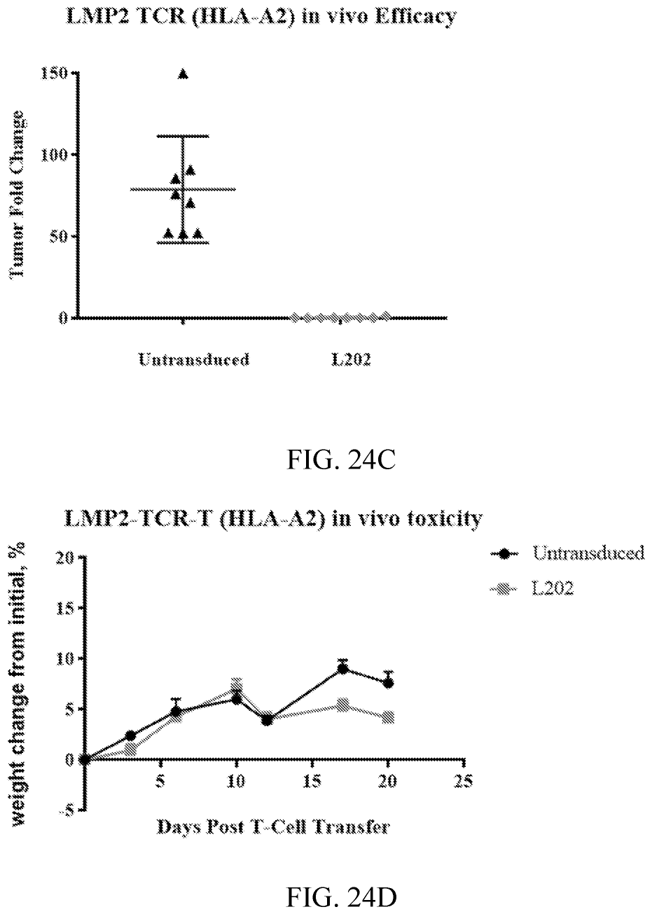

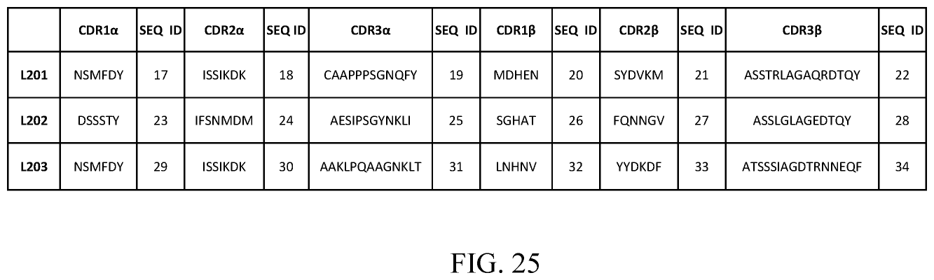

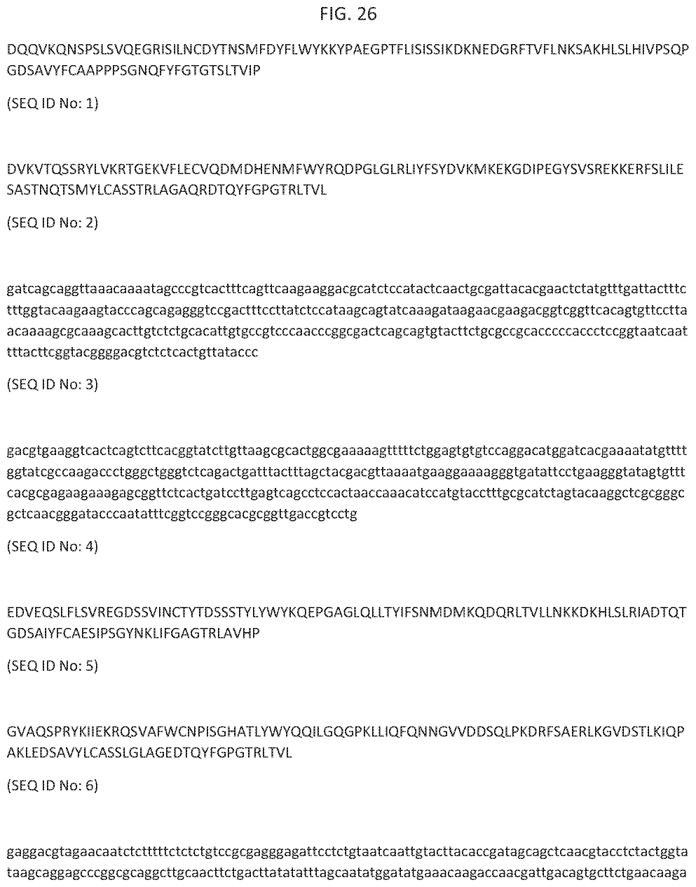

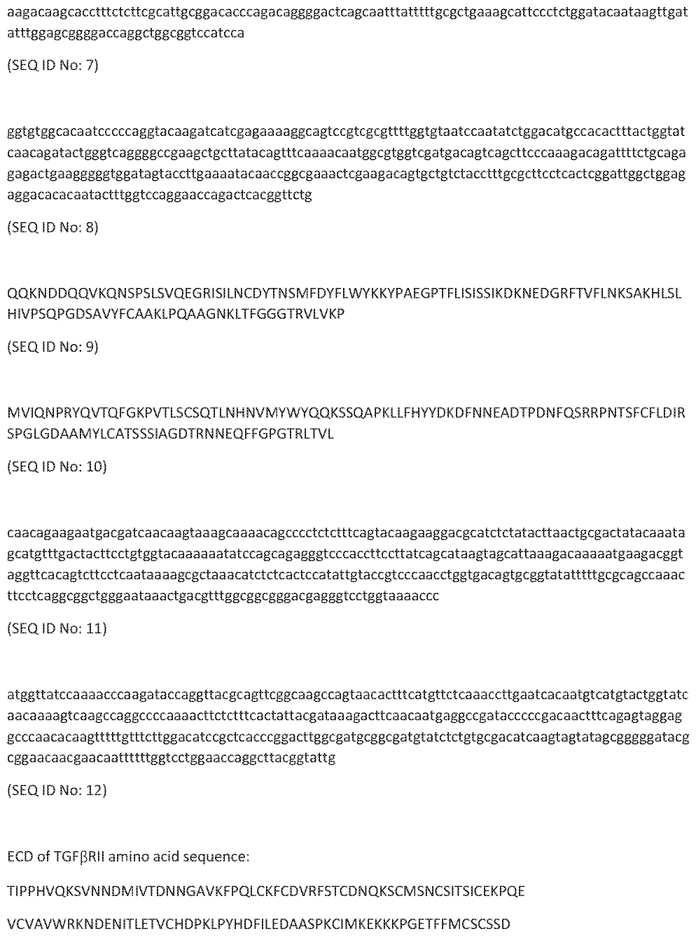

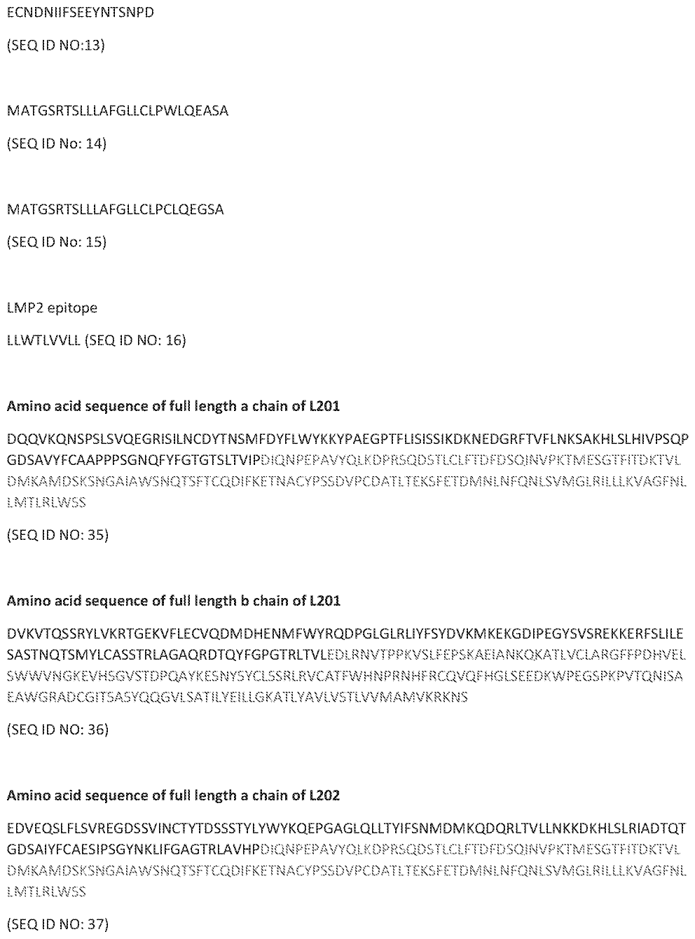

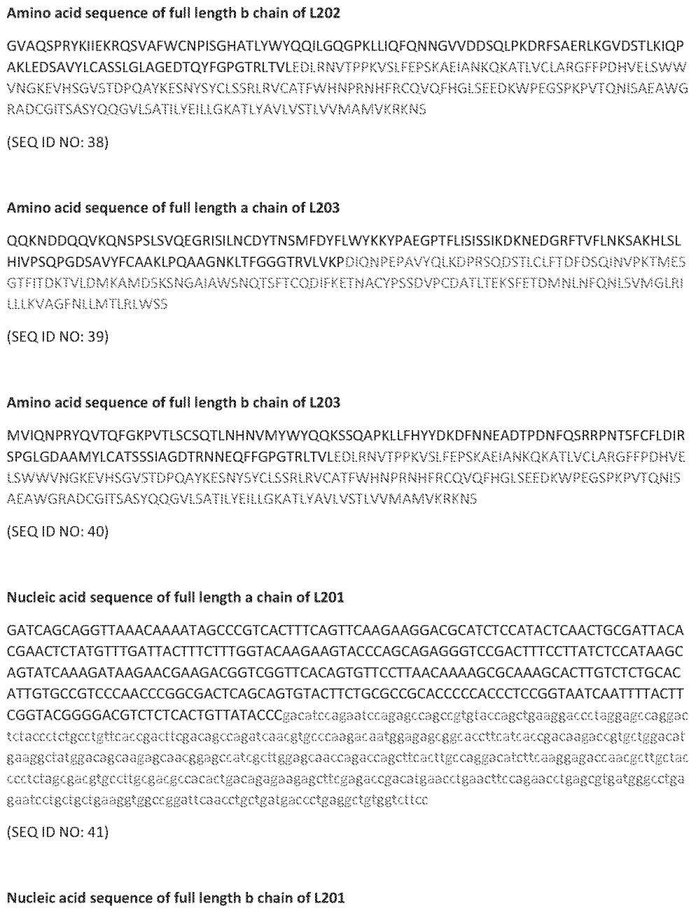

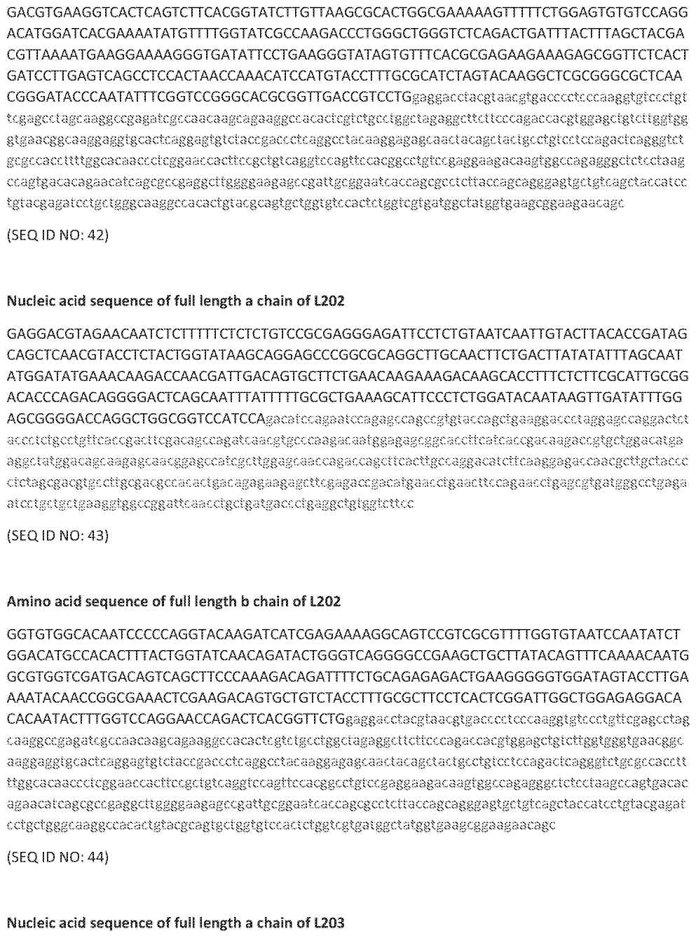

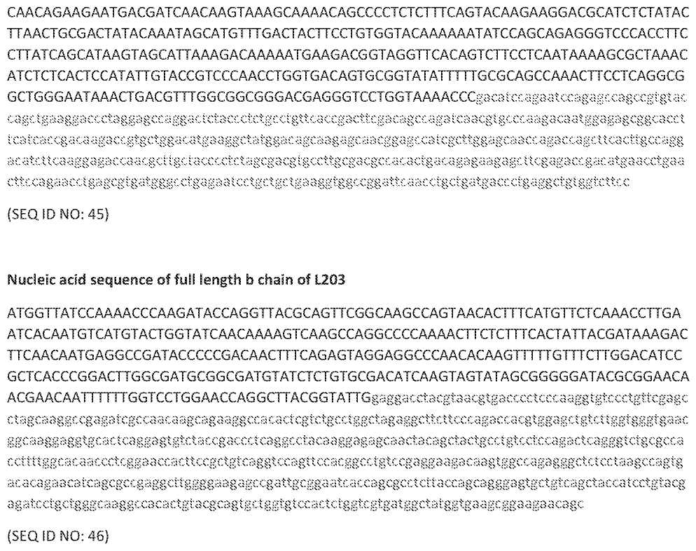

Tumor volumes were measured on the indicated days and plotted individually (FIG. 24A) or as the mean for each group (FIG. 24B). Tumor fold changes (FIG. 24C) were calculated and plotted as the (tumor volume on day 20)/(tumor volume on day 0). Animal body weight changes were calculated as percentages based on initial animal weights on day 0 (FIG. 24D). Together, these results demonstrate robust antitumor efficacy of L202 TCR-T cells with no evidence of apparent toxicity.

### Prophetic Method

6-8-week-old female NSG mice will be inoculated with 5.0×10⁶ A375-pep-HLA-A2 melanoma cells subcutaneously in the right flank. 9 days later, on study Day 0, animals will be sorted into groups based on tumor volume with each group bearing an average tumor volume of 35 mm³. On study Day 0 animals will be intravenously injected with 1e6 untransduced cells or TCR-T cells transduced with the following constructs: 1) L202; 2) L202-PD1; 3) L202-TGFβRII; 4) L202-PD1-TGFβRII. These injections will be repeated 7 days later, on study Day 6. Tumor volumes and animal weights will then be measured every 2 days until day 20, when the experiment will be terminated.

In contrast with 10×10⁶ L202 cells which completely eliminated A375 tumors (FIG. 24B), we expect treatment with 1×10⁶ L202 cells to modestly inhibit tumor growth in vivo. Based on the known capacity of PD1 to drive the growth of A375 melanoma (cite PMID: 26359984), we expect the addition of anti-PD1 to produce a stronger reduction in mouse tumor burdens in comparison with L202 alone (group 2 vs. group 1). A further additive or synergistic effect will be determined by examining TGFβ antagonism with and without anti-PD1 (groups 4 and 3, respectively, vs. groups 1 and 2). Together, we expect these experiments to provide proof-of-principle that combining TCR-T cell therapy with immune checkpoint inhibition and/or TGFβ blockade provides quantitatively greater antitumor efficacy, thereby facilitating the use of smaller dosing regimens.

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed, Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 Jul. 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here. The compositions and methods of the present invention are not limited to variants of the exemplary sequences disclosed herein but include those having at least 90%, at least 95% and at least 99% identity to an exemplary sequence disclosed herein.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

We claim:

1. An engineered T cell, comprising:
a nucleic acid encoding an anti-LMP2 TCR wherein the anti-LMP2 TCR comprises genetically engineered T cell receptor that specifically binds to LMP2 in a tumor.

2. The engineered T cells of claim 1, wherein the anti-LMP2 TCR comprises alpha chain CDR1 (position 27-32), CDR2 (position 50-56), CDR3 (position 90-101) of amino acid SEQ ID NO:1 and beta chain CDR1 (position 27-31), CDR2 (position 49-54), CDR3 (position 92-106) of amino acid SEQ ID NO:2 respectively.

3. The engineered T cell of claim 1, wherein the anti-LMP2 TCR comprises an alpha chain variable domain comprising SEQ ID NO:1 and a beta chain variable domain comprising SEQ ID NO:2.

4. The engineered T cell of claim 1, wherein the nucleic acid comprises the SEQ ID NO:3 and SEQ ID NO:4.

5. The engineered T cells of claim 1, wherein the anti-LMP2 TCR comprises alpha chain CDR1 (position 25-30), CDR2 (position 48-54), CDR3 (position 89-100) of amino acid SEQ ID NO:5 and beta chain CDR1 (position 25-29), CDR2 (position 47-52), CDR3 (position 91-103) of amino acid SEQ ID NO:6 respectively.

6. The engineered T cell of claim 1, wherein the anti-LMP2 TCR comprises an alpha chain variable domain comprising SEQ ID NO:5 and a beta chain variable domain comprising SEQ ID NO:6.

7. The engineered T cell of claim 1, wherein the nucleic acid comprises the SEQ ID NO:7 and SEQ ID NO:8.

8. The engineered T cells of claim 1, wherein the anti-LMP2 TCR comprises an alpha chain CDR1 (position 32-37), CDR2 (position 55-61), CDR3 (position 96-108) of amino acid SEQ ID NO:9 and beta chain CDR1 (position 25-29), CDR2 (position 47-52), CDR3 (position 90-105) of amino acid SEQ ID NO:10 respectively.

9. The engineered T cell of claim 1, wherein the anti-LMP2 TCR comprises an alpha chain variable domain comprising SEQ ID NO:9 and a beta chain variable domain comprising SEQ ID NO:10.

10. The engineered T cell of claim 1, wherein the nucleic acid comprises the SEQ ID NO:11 and SEQ ID NO:12.

11. The engineered T cell of claim 1, wherein the anti-LMP2 TCR is constitutively expressed.

12. The engineered T cell of claim 1, further comprising an inhibitory protein that reduces function or expression of inhibitory receptors in a tumor.

13. The engineered T cells of claim 12, wherein the inhibitory protein is an immune checkpoint inhibitor.

14. A pharmaceutical composition, comprising the engineered T cell of any of claims 1-13 and a pharmaceutically acceptable carrier.

15. A method for treating cancer comprising administering to a subject in need thereof, a therapeutically effective amount of the cell of claim 14.

16. The method of claim 15 wherein the cancer is nasopharyngeal carcinoma, Hodgkin's lymphoma, Burkitt's lymphoma, or stomach cancer.

17. The method of claim 16, further comprising administering to the subject a therapeutically effective amount of an existing therapy comprising chemotherapy or radiation.

18. The method of claim 17, wherein the cell and the existing therapy are administered sequentially or simultaneously.

19. An engineered T cell, comprising:
a nucleic acid encoding (a) a genetically engineered T cell receptor that specifically binds to an antigen in a tumor;
(b) an inhibitory protein that reduces function or expression of an immune checkpoint in a tumor; and
(c) a protein that binds to a member of the transforming growth factor beta family (TGF-β).

20. The engineered T cell of claim 19, wherein the immune checkpoint comprises one or more of PD1, PD-L1 and CTLA-4.

21. The engineered T cell of claim 19, wherein the antigen in a tumor comprises human papillomavirus (HPV) or Epstein-Barr virus (EBV) antigen.

22. The engineered T cell of claim 21, wherein the genetically engineered T cell receptor is an anti-LMP2 TCR.

23. The engineered T cell of claim 21, wherein the genetically engineered T cell receptor is an anti-E6 TCR.

24. The engineered T cell of any one of claims 19-23, wherein the binding protein comprises the extracellular domain of TGFβRII.

25. The engineered T cell of claim 22, wherein the anti-LMP2 TCR comprises an alpha chain variable domain of SEQ ID NO:1 and a beta chain variable domain of SEQ ID NO:2.

26. The engineered T cell of claim 22, wherein the nucleic acid encoding the genetically engineered antigen receptor comprises SEQ ID NO:3 and SEQ ID NO:4.

27. The engineered T cell of any one of claims 19-26, wherein the genetically engineered TCR is constitutively expressed.

28. The engineered T cell of claim 27, wherein the binding protein targeting TGF-β is constitutively expressed.

29. A vector comprising the nucleic acid according to claim 19.

30. The vector of claim 29, wherein the vector is a retroviral vector.

31. A pharmaceutical composition, comprising the engineered T cell of any of claims 19-28 and a pharmaceutically acceptable carrier.

32. A method for treating cancer comprising administering to a subject in need thereof, a therapeutically effective amount of the cell of claim 31.

33. The method of claim 32, wherein the cancer is nasopharyngeal carcinoma, Hodgkin's lymphoma, Burkitt's lymphoma, or stomach cancer.

34. The method of claim 32, wherein the cancer is cervical, anal, oropharyngeal, or reproductive organ cancers.

35. The method of any one of claims 33-34, further comprising administering to the subject a therapeutically effective amount of an existing therapy comprising chemotherapy or radiation.

36. The method of claim 35, wherein the cell and the existing therapy are administered sequentially or simultaneously.

37. The engineered T cells in any one of claims 19-28, wherein the tumor is a virus-associated tumor.

38. A T cell receptor (TCR) or antigen-binding fragment thereof, comprising an alpha chain comprising a variable alpha (Va) region and a beta chain comprising a variable beta (Vb) region, wherein:
(1) the Va region comprises a complementarity determining region 1 (CDR1), a complementarity determining region 2 (CDR2), and a complementarity determining region 3 (CDR3), comprising CDR1, CDR2, and CDR3 of SEQ ID NO:1, respectively, and the Vb region comprises a CDR1, a CDR2, and a CDR3, comprising CDR1, CDR2, and CDR3 of SEQ ID NO: 2, respectively;
(2) the Va region comprises a CDR1, a CDR2, and a CDR3, comprising CDR1, CDR2, CDR3 of SEQ ID NO: 5, respectively, and the Vb region comprises a CDR1, a CDR2, and a CDR3, comprising the amino acid sequences of CDR1, CDR2, and CDR3 of SEQ ID NO: 6, respectively;
or
(3) the Va region comprises a CDR1, a CDR2, and a CDR3, comprising CDR1, CDR2, CDR3 of SEQ ID NO: 9, respectively, and the Vb region comprises a CDR1, a CDR2, and a CDR3, comprising the amino acid sequences of CDR1, CDR2, and CDR3 of SEQ ID NO: 10, respectively.

39. The T cell receptor (TCR) or antigen-binding fragment thereof of claim 38, wherein
(1) the Va region comprises a CDR1, a CDR2, and a CDR3, comprising amino acids of SEQ ID NOs: 17-19, respectively, and the Vb region comprises a CDR1, a CDR2, and a CDR3, comprising amino acids of SEQ ID NOs: 20-22, respectively;
(2) the Va region comprises a CDR1, a CDR2, and a CDR3, comprising amino acids of SEQ ID NOs: 23-25, respectively, and the Vb region comprises a CDR1, a CDR2, and a CDR3, comprising amino acids of SEQ ID NOs: 26-28, respectively; or
(3) the Va region comprises a CDR1, a CDR2, and a CDR3, comprising amino acids of SEQ ID NOs: 29-31, respectively, and the Vb region comprises a CDR1, a CDR2, and a CDR3, comprising amino acids at position 25-29, amino acids of SEQ ID NOs: 32-34, respectively.

40. The T cell receptor (TCR) or antigen-binding fragment thereof of claim 38, wherein:
the Va region comprises the amino acid sequence set forth in any of SEQ ID NOs: 1, 5, or 9, or an amino acid sequence that has at least 80%, 85%, 90%, 91%, 92%, 93%, 94%, 95%, 96%, 97%, 98%, or 99% sequence identity thereto; and
the Vb region comprises the amino acid sequence set forth in any of SEQ ID NOs: 2, 6, or 10, or an amino acid sequence that has at least 80%, 85%, 90%, 91%, 92%, 93%, 94%, 95%, 96%, 97%, 98%, or 99% sequence identity thereto.

41. The TCR or antigen-binding fragment thereof of any of claims 38-40, wherein the TCR or antigen-binding fragment thereof binds to or recognizes a peptide epitope of LMP2 (LLWTLVVLL) (SEQ ID NO: 16).

42. The TCR or antigen-binding fragment thereof of any of claims 38-41, wherein, the TCR or antigen-binding fragment thereof, when expressed on the surface of a T cell, stimulates cytotoxic activity against a target cancer cell, optionally wherein the target cancer cell contains EBV DNA sequences or expresses LMP2.

43. A vector comprising a nucleic acid encoding TCR or antigen-binding fragment thereof of any of claims 38-42.

44. The vector of claim 43, wherein the vector is an expression vector, a viral vector, a retroviral vector, or a lentiviral vector.

45. An engineered cell comprising the vector of any of claims 43-44.

46. An engineered cell, comprising the TCR or antigen-binding fragment thereof of any of claims 38-42.

47. The engineered cell of claim 46, wherein the TCR or antigen binding fragment thereof is heterologous to the cell.

48. The engineered cell of any of claims 45-47, wherein the engineered cell is a cell line.

49. The engineered cell of any of claims 45-47, wherein the engineered cell is a primary cell obtained from a subject (e.g., a human subject).

50. The engineered cell of any of claims 45-47, wherein the engineered cell is a T cell.

51. The engineered cell of claim 50, wherein the T cell is CD8+.

52. The engineered cell of claim 50, wherein the T cell is CD4+.

53. A method for producing the engineered cell, comprising introducing a vector of claim 43 or 44 into a cell in vitro or ex vivo.

54. The method of claim 53, wherein the vector is a viral vector and the introducing is carried out by transduction.

55. A method of treating a disease or a disorder, comprising administering the engineered cell of any of claims 45-52 to a subject having a disease or disorder associated with EBV.

56. The method of claim 55, wherein the disease or disorder associated with EBV is a cancer.

57. A method of treating a tumor in a subject, the method comprising
administering to the subject in need thereof
(a) an engineered T cell, comprising: a nucleic acid encoding a TCR or antigen-binding fragment thereof that specifically binds to an antigen in a tumor; and
(b) either one of both of a checkpoint inhibitor or a protein that binds to a member of the transforming growth factor beta family (TGF-β).

58. A method of treating a tumor in a subject, the method comprising
administering to the subject in need thereof
an engineered T cell, comprising: a nucleic acid encoding
(a) a TCR or antigen-binding fragment thereof that specifically binds to an antigen in a tumor; and
(b) a bifunctional trap protein that targets a checkpoint inhibitor and a member of the transforming growth factor beta family (TGF-β).

59. The method of claim 57 or 58, wherein the tumor is EBV-induced tumor or HPV-induced tumor.
